(19) United States
(12) Patent Application Publication
(10) Pub. No.: US 2024/0018606 A1
(43) Pub. Date: Jan. 18, 2024

(54) NEW NATIVE CLUBROOT RESISTANCE IN RAPESEED BRASSICA NAPUS

(71) Applicant: NPZ INNOVATION GMBH, Hollsee (DE)

(72) Inventor: Steffen RIETZ, Haby (DE)

(21) Appl. No.: 18/036,133

(22) PCT Filed: Dec. 20, 2021
(86) PCT No.: PCT/EP2021/086738

§ 371 Date: May 9, 2023

(30) Foreign Application Priority Data
Dec. 21, 2020 (EP) 20216193.1

(51) Int. Cl. C12Q 1/6895; C12N 15/82

(52) U.S. Cl. CPC C12Q 1/6895; C12N 15/8279; C12Q 2600/13

(57) ABSTRACT

The present invention provides methods for identifying or selecting a Brassica plant cell, Brassica plant or part thereof, or a Brassica seed comprising a QTL that confers resistance against clubroot disease. The present invention further provides a nucleic acid comprising a QTL that confers resistance against clubroot disease as well as markers associated therewith, proteins encoded by the nucleic acid, plants or parts thereof, plant cells or seeds comprising the QTL and uses thereof.

### FIELD OF THE INVENTION

The present invention provides methods for identifying or selecting a Brassica plant cell, Brassica plant or part thereof, or a Brassica seed comprising a QTL that confers resistance against clubroot disease.

The present invention further provides a nucleic acid comprising a QTL that confers resistance against clubroot disease as well as markers associated therewith, proteins encoded by the nucleic acid, plants or parts thereof, plant cells or seeds comprising the QTL and uses thereof. The QTL was identified on chromosome A06 in the genome of a Brassica napus plant.

### BACKGROUND OF THE INVENTION

Clubroot disease is a major disease of many cruciferous species in the world including important crops like oilseed rape, cabbage, radish, and turnip rape and thus is of high economic significance. The disease is caused by Plasmodiophora brassicae W., a eukaryotic, unicellular microorganism living in the soil. In the presence of a host plant zoospores of Plasmodiophora brassicae infect the roots and form plasmodia for its proliferation (Schwelm et al., 2015). A susceptible plant responds with hypertrophic growth of root tissue resulting in galls along the primary and secondary roots up to complete transition from root to gall structure. At high infection incidence the plants are strongly impaired in shoot growth and seed production or a complete plant loss can occur, which severely impacts yield.

In the field this pathogen mainly spreads by the movement of soil, e.g. particles attached to farm machines or soil erosion. In addition, resting spores can survive in the soil for many years and thus Plasmodiophora brassicae inoculum builds up with tighter rotation of Brassica crops. On the other hand, no chemical treatment is available to control Plasmodiophora brassicae in the field and the only way to effectively cope with this disease is to grow crop varieties with genetic resistance against Plasmodiophora brassicae. Although highly efficient, this resistance protects against certain races of Plasmodiophora brassicae only and thus is considered race-specific.

Sources of genetic resistance have been detected in Brassica rapa L., Brassica oleracea L., Brassica nigra L. and Brassica napus L. (reviewed in Piao et al., 2009; Rahman et al., 2014). Whereas native resistance against Plasmodiophora brassicae inside Brassica oleracea and Brassica napus species are rare and often predisposed by minor quantitative loci (Manzanares-Dauleux et al., 2009; Tomita et al., 2013; Dakouri et al., 2018; Aigu et al., 2020), some European fodder turnips (Brassica rapa) are known to possess strong resistance against many pathotypes of Plasmodiophora brassicae. Genetic Crosses with these resistant fodder turnips were therefore undertaken in the past to transfer these resistances to related crop species. It is thought that clubroot resistance (CR) of fodder turnips is mediated by a combination of at least three loci in the genome, called A, B and C (Buczacki et al., 1975). Previous genetic analyses detected several dominant resistance loci in the Brassica rapa genome and the majority of those are located on the A03 and A08 chromosomes (Chen et al., 2013; Chu et al., 2014; Kato et al., 2013; Pang et al., 2018; Yu et al., 2017; Laila et al., 2019). Fine mapping and molecular analysis of two genes conferring CR revealed homology of the corresponding proteins to Toll Interleukin 1 Receptor-Nucleotide Binding-Leucine Rich Repeat (TIR-NB-LRR) type proteins that are known to trigger plant defense responses in various plant microbe interactions (Ueno et al., 2012; Hatakeyama et al., 2013, 2017).

In rapeseed (Brassica napus) resistant varieties today originate almost from NPZ variety MENDEL (Norddeutsche Pflanzenzucht Hans-Georg Lembke KG (NPZ), Germany). MENDEL itself inherited the resistance from Brassica rapa fodder turnip through extensive backcrossing and consecutive selection of the resistance with biotests and molecular marker. MENDEL confers a race-specific resistance that is incomplete in comparison to the CR traits available in fodder turnips. Expansion of rapeseed cropping area over the last 20 years revealed an increasing number of sites with Plasmodiophora brassicae pathotypes able to infect the MENDEL-resistance. A significant extension of the resistance to a range of avirulent Plasmodiophora brassicae isolates of MENDEL was recently achieved by the CR trait CRE1 (NPZ, Rietz et al., 2019). However, CRE1 still can be infected by further pathotypes of Plasmodiophora brassicae which in turn are not able to infect CR of certain fodder turnips.

In general, once Plasmodiophora brassicae races able to infect plant resistance have built up in a field, growing of the respective crop is no more profitable on this site. Thus, the provision of new and complex CRs is a prerequisite for the continuance of rapeseed in cropping areas worldwide.

The aim of the current invention is to (1) generate a Brassica napus line with a CR that significantly extends the MENDEL and CRE1 resistances in terms of race-specificity and (2) describe a method for identifying lines carrying new CR. Such line would be resistant against different Plasmodiophora brassicae isolates able to infect the MENDEL or CRE1 resistance.

### SUMMARY OF THE INVENTION

Methods for Identifying or Selecting a Brassica Plant

In one aspect, the present invention provides a method for identifying or selecting a Brassica plant cell, Brassica plant or part thereof, or a Brassica seed comprising a QTL that confers resistance against clubroot disease, the method comprising
(i) detecting a QTL in the genome of the Brassica plant cell, Brassica plant or part thereof, or Brassica seed between a marker according to SEQ ID NO: 1 and a marker according to SEQ ID NO: 2; or
(ii) detecting a QTL in the genome of the Brassica plant cell, Brassica plant or part thereof, or Brassica seed linked to a marker according to any one of SEQ ID NO: 3 to SEQ ID NO: 29; or
(iii) detecting a QTL in the genome of the Brassica plant cell, Brassica plant or part thereof, or Brassica seed localizing within a genomic region, wherein the genomic region corresponds to position 3759027 to 8593901 of chromosome A06 of the Darmor-bzh reference genome.

SEQ ID NO: 1 corresponds to a nucleic acid sequence comprising a Single Nucleotide Polymorphism (SNP) that is linked to the QTL. SEQ ID NO: 2 corresponds to a nucleic acid sequence comprising a SNP that is linked to the QTL. Each of SEQ ID NO: 3 to SEQ ID NO: 29 corresponds to a nucleic acid sequence comprising a SNP that is linked to the QTL. The SNP is present in the genome sequence of a Brassica plant that comprises the QTL of the present invention in comparison to the corresponding genome sequence of a Brassica plant that does not comprise the QTL of the present invention.

The nucleic acid sequences of SEQ ID NO: 1 to SEQ ID NO:29 are depicted in Table 2. The nucleotide at the 3′ end is the SNP position. The nucleotide indicated at this position in Table 2 is present in the genome sequence of a Brassica plant that comprises the QTL of the present invention.

In a preferred embodiment, the method for identifying or selecting a Brassica plant cell, Brassica plant or part thereof, or a Brassica seed comprising a QTL that confers resistance against clubroot disease, comprises detecting a QTL in the genome of the Brassica plant cell, Brassica plant or part thereof, or Brassica seed linked to the marker according to SEQ ID NO: 3.

Presently, the genome of the Brassica napus winter oilseed cultivar Darmor-bzh serves as reference genome. The specified nucleotide positions indicate the position of the allele in the Darmor-bzh reference genome. Accordingly, for other Brassica genomes, it is referred to the respective corresponding positions. Hence, it is referred to the position in the respective Brassica genome that corresponds to certain specified positions in the Darmor-bzh reference genome.

In another aspect, the present invention provides a method for identifying or selecting a Brassica plant cell, Brassica plant or part thereof, or a Brassica seed comprising a QTL that confers resistance against clubroot disease, the method comprising
(i) detecting a QTL in the genome of the Brassica plant cell, Brassica plant or part thereof, or Brassica seed corresponding to a QTL localizing on chromosome A06 of Brassica napus grown from seeds deposited under NCIMB 43700 between a marker according to SEQ ID NO: 1 and a marker according to SEQ ID NO: 2; or
(ii) detecting a QTL in the genome of the Brassica plant cell, Brassica plant or part thereof, or Brassica seed corresponding to a QTL localizing on chromosome A06 of Brassica napus grown from seeds deposited under NCIMB 43700 linked to a marker according to any one of SEQ ID NO: 3 to SEQ ID NO: 29.

In one embodiment, method involves the steps of
(a) obtaining a sample from the Brassica plant cell, the Brassica plant or part thereof, or the Brassica seed;
(b) isolating genomic DNA from the sample; and
(c) detecting the presence of the QTL in the genomic DNA.

In one embodiment, detection of the QTL involves the detection of one or more markers linked to the QTL. For example, the detection may involve one, two, three, four, five or more markers that are linked to the QTL. The detection of the QTL may involve the detection of two or more markers that are linked to the QTL and that are flanking the QTL.

Isolation of the genomic DNA may be carried out by any suitable method. The detection of the one or more markers may also involve any method suitable for such detection. In one embodiment, the detection of the one or more markers occurs by PCR, hybridization, KASP assay, SNP analysis, genotyping, nucleic acid sequencing, next generation sequencing, nuclease based detection, by using an antibody, by using nucleic acid probes or by DNA chip technology. In a preferred embodiment, the detection of the one or more markers occurs by KASP assay.

In one embodiment, the one or more markers linked to the QTL are within 50 cM of the QTL, within 45 cM, within 40 cM, within 35 cM, within 30 cM, within 25 cM, within 20 cM, within 15 cM, within 10 cM or within 5 cM of the QTL. In a preferred embodiment, the one or more markers linked to the QTL are within 10 cM of the QTL.

In one embodiment, the resistance against clubroot disease is dominant.

The Brassica plant cell, Brassica plant or part thereof, or Brassica seed may be of any Brassica species. In one embodiment, the Brassica plant cell, Brassica plant or part thereof, or Brassica seed is Brassica napus. In another embodiment, the Brassica plant cell, Brassica plant or part thereof, or Brassica seed is Brassica rapa. In yet another embodiment, the Brassica plant cell, Brassica plant or part thereof, or Brassica seed is Brassica juncea. In yet another embodiment, the Brassica plant cell, Brassica plant or part thereof, or Brassica seed is Brassica carinata. In yet another embodiment, the Brassica plant cell, Brassica plant or part thereof, or Brassica seed is Brassica oleracea.

In a preferred embodiment, the Brassica plant cell, Brassica plant or part thereof, or Brassica seed is Brassica napus.

In one embodiment, the method of the invention is for determining the zygosity status of the Brassica plant cell, the Brassica plant or part thereof, or the Brassica seed with regard to the QTL. The zygosity status with regard to the QTL may be homozygous, heterozygous, hemizygous or nullizygous.

In one embodiment, the QTL is linked to a marker according to any one of SEQ ID NO:1, 2, 3, 4, 7, 9, 10, 11, 12, 14, 16, 17, 18, 20, 23, 24, 25, 27 or 29.

Nucleic Acids and Proteins

In one aspect, the invention provides a nucleic acid comprising a QTL that confers resistance against clubroot disease localizing within a genomic region of Brassica napus grown from seeds deposited under NCIMB 43700, wherein the genomic region corresponds to position 3759027 to 8593901 of chromosome A06 in the Darmor-bzh reference genome.

In one embodiment, the invention provides a nucleic acid comprising a QTL that confers resistance against clubroot disease, wherein the QTL has at least 70% sequence identity, at least 75% sequence identity, at least 80% sequence identity, at least 85% sequence identity, at least 90% sequence identity, at least 95% sequence identity, at least 98% sequence identity, at least 99% sequence identity with a QTL localizing within a genomic region of Brassica napus grown from seeds deposited under NCIMB 43700, wherein the genomic region corresponds to position 3759027 to 8593901 of chromosome A06 of the Darmor-bzh reference genome.

In yet another aspect, the invention provides a nucleic acid comprising a QTL that confers resistance against clubroot disease localizing within a genomic region of the genome of a Brassica plant
(a) flanked by a marker according to SEQ ID NO: 1 and a marker according to SEQ ID NO: 2, or
(b) linked to a marker according to any one of SEQ ID NO: 3 to SEQ ID NO: 29.

In yet another aspect, the invention provides a nucleic acid comprising a QTL that confers resistance against clubroot disease, wherein the QTL has at least 70% sequence identity, at least 75% sequence identity, at least 80% sequence identity, at least 85% sequence identity, at least 90% sequence identity, at least 95% sequence identity, at least 98% sequence identity, at least 99% sequence identity with a QTL that confers resistance against clubroot disease localizing within a genomic region of the genome of a Brassica plant
(a) flanked by a marker according to SEQ ID NO: 1 and a marker according to SEQ ID NO: 2, or
(b) linked to a marker according to any one of SEQ ID NO: 3 to SEQ ID NO: 29.

In one such embodiment, the Brassica plant is grown from seeds deposited under NCIMB 43700.

In one aspect, the invention provides a nucleic acid comprising an open reading frame encoding a protein conferring resistance against clubroot disease, wherein the encoded protein sequence corresponds to an encoded protein sequence localizing within a genomic region of Brassica napus grown from seeds deposited under NCIMB 43700, wherein the genomic region corresponds to position 3759027 to 8593901 of chromosome A06 in the Darmor-bzh reference genome.

The invention also provides a protein encoded by a nucleic acid of the invention. The invention further provides an antibody or an antibody fragment directed against the protein of the invention. The antibody or antibody fragment may be any type of antibody that specifically binds the protein of the invention.

In one such embodiment, the nucleic acid or the protein of the invention confers resistance against clubroot disease.

In one embodiment, the method for identifying or selecting a Brassica plant cell, Brassica plant or part thereof, or a Brassica seed comprising a QTL that confers resistance against clubroot disease involves a nucleic acid of the invention or an antibody of the invention.

In a further aspect, the invention provides a nucleic acid for detecting the QTL of the invention. In one embodiment, the nucleic acid is for detecting a marker according to any one of SEQ ID NO: 1 to SEQ ID NO: 29. In one embodiment, the nucleic acid comprises a nucleic acid sequence according to any one of SEQ ID NO: 1 to SEQ ID NO: 29. In one embodiment, the nucleic acid comprises at least 15 nucleotides starting from the 3′ end of the nucleic acid sequence according to any one of SEQ ID NO: 1 to SEQ ID NO: 29. In one embodiment, the nucleic acid comprises at least 20 nucleotides starting from the 3′ end of the nucleic acid sequence according to any one of SEQ ID NO: 1 to SEQ ID NO: 29.

In a further aspect, the invention provides the use of a nucleic acid for detecting the QTL of the invention. In one embodiment, the nucleic acid comprises a nucleic acid sequence according to any one of SEQ ID NO: 1 to SEQ ID NO: 29. In one embodiment, the nucleic acid comprises at least 15 nucleotides starting from the 3′ end of the nucleic acid sequence according to any one of SEQ ID NO: 1 to SEQ ID NO: 29. In one embodiment, the nucleic acid comprises at least 20 nucleotides starting from the 3′ end of the nucleic acid sequence according to any one of SEQ ID NO: 1 to SEQ ID NO: 29.

Plant Cell, Plant or Part Thereof, Seed and Descendants

In one aspect, the invention provides a plant cell, plant or part thereof, or a seed identified or selected by a method of the invention.

In one such embodiment, the plant cell, the plant, or part thereof, or the seed is homozygous or heterozygous for the nucleic acid.

The plant cell, plant, or part thereof, or a seed identified or selected by a method of the invention may be of any Brassica species. In one embodiment, the invention provides a Brassica napus plant cell, a Brassica napus plant or part thereof, or a Brassica napus seed comprising a nucleic acid of the invention or a protein of the invention.

In another aspect, the invention provides a Brassica napus plant cell, a Brassica napus plant or part thereof grown from seeds deposited under NCIMB 43700.

In a further aspect, the invention also provides a descendant of the plant of the invention. In one embodiment, the descendant is obtained by crossing. In another embodiment, the descendant is obtainable by crossing.

The descendant is for example obtained by or obtainable by crossing a first Brassica plant with a second Brassica plant, wherein one of the plants was grown from seeds of which a representative sample was deposited under NCIMB accession number NCIMB 43700.

The descendant is for example obtained by or obtainable by crossing any Brassica plant, in particular any Brassica napus plant, carrying the nucleic acid of the invention as present in seeds of which a representative sample was deposited under NCIMB accession number NCIMB 43700.

In one embodiment, the descendant comprises a nucleic acid or a protein of the invention.

In a further embodiment, the plant cell, the plant, or part thereof is resistant against clubroot disease. In a further embodiment, a plant grown from the seed of the invention is resistant against clubroot disease.

In another aspect, the invention also provides a harvested part of a plant of the invention. In one embodiment, the harvested part is a feed product.

In another aspect, the invention provides a population of plant cells, a population of plants or parts thereof, or a population of seeds identified or selected by a method of the invention. All of the embodiments described herein equally apply to the population of plant cells, the population of plants or parts thereof, and the population of seeds.

Uses and Further Methods

In one aspect, the invention also provides the use of a plant of the invention or a plant grown from a seed of the invention for breeding, wherein the breeding method is conventional breeding, hybrid breeding, pedigree breeding, crossing, self-pollination, doubling haploidy, single seed descent, backcrossing, somatic hybridization, protoplast fusion or breeding by genetic transformation or genome editing, targeted mutagenesis or non-targeted mutagenesis. In a preferred embodiment, the breeding method is crossing.

In one aspect, the invention provides a method for increasing resistance against clubroot disease in a plant, wherein the method comprises a step of introducing a nucleic acid of the invention into the plant.

In another aspect, the invention provides the use of the nucleic acid of the invention in a method for increasing the resistance against clubroot disease in a plant.

In one embodiment, the plant is a Brassica species. In a preferred embodiment, the plant is Brassica napus.

In one embodiment, the method involves genome editing, genetic transformation, targeted mutagenesis or non-targeted mutagenesis. In a preferred embodiment, the method involves genetic transformation. In a further embodiment, the method involves breeding. In a preferred embodiment, the method involves crossing.

In another aspect, the invention provides a method for identifying a marker linked to a QTL that confers resistance against clubroot disease, wherein the QTL is localized
(i) within a genomic region of a Brassica plant between a marker according to SEQ ID NO: 1 and a marker according to SEQ ID NO: 2; or
(ii) within a genomic region of a Brassica plant linked to a marker according to any one of SEQ ID NO: 3 to SEQ ID NO: 29, or
(iii) within a genomic region of a Brassica plant corresponding to position 3759027 to 8593901 of the Darmor-bzh reference genome.

In one embodiment, the marker is within 50 cM, within 45 cM, within 40 cM, within 35 cM, within cM, within 25 cM, within 20 cM, within 15 cM, within 10 cM or within 5 cM of the QTL. In a preferred embodiment, the marker is within 10 cM of the QTL.

In a further aspect, the invention provides a marker identified by a method of the invention. In one embodiment, the identified marker is a SNP. In one embodiment, the marker is located on chromosome A06 between a marker according to SEQ ID NO: 1 and a marker according to SEQ ID NO: 2.

In a further aspect, the invention provides a method for transferring a resistance to clubroot disease to a descendant of a Brassica plant comprising the steps of:
(i) obtaining a first Brassica plant that is resistant to clubroot disease,
(ii) crossing the first Brassica plant of step (i) with a second Brassica plant,
(iii) selecting a descendant of the first and second Brassica plant that is resistant to clubroot disease according to the method of the invention,
wherein the first Brassica plant comprises a nucleic acid of the invention.

In a further aspect, the invention provides a method for obtaining oil or meal products from a Brassica seed comprising a nucleic acid of the invention or from a Brassica seed, wherein a representative of said seed is deposited under NCIMB 43700, wherein the method comprises the steps of
(i) crushing the Brassica seed, and
(ii) extracting the oil or meal products from the brassica seed.

Steps involved in seed crushing may include any suitable method including dehulling, flacking, cooking, pressing, extraction, distillation, refining, and desolvantisation.

In a further aspect, the invention provides oil or meal products obtained by a method of the invention.

In a further aspect, the invention provides a crushed Brassica seed comprising the nucleic acid of the invention. In one embodiment, a representative of said seed is deposited under NCIMB 43700.

Plants of the invention can be used for the production of seeds, as a cover crop to prevent soil erosion, for the production of biomass, as a weed suppressor, for forage and grazing by livestock, for forage by honeybees, or to improve soil tilth. In a preferred embodiment, the plants of the invention are used for the production of seeds. The oil of the invention can be used as a food product, as biofuel or biolubricant. The meal of the invention can be used as animal feed or as a soil fertilizer.

### BRIEF DESCRIPTION OF THE FIGURES

FIG. 1 shows a genome-wide QTL plot with SNP marker polymorphic inside the Brassica napus F2 mapping population Nr. 4 (n=72). Lod values indicate linkage of SNP to the new CR trait.

FIG. 2A shows a QTL plot of Brassica napus Chromosome A06, showing the position of the SNP linked to the new CR trait in the F2 population Nr. 4 (n=72).

FIG. 2B shows the marker effect of most significant chip-marker in this region. A=dominant resistance allele.

FIG. 3 shows the results of a resistance test with five isolates of Plasmodiophora brassicae (P.b. #1 to #5) on a Brassica differential set including new CR. Numbers on the x-axis represent different plant genotypes: 1=new clubroot resistant line with QTL on chromosome A06; 2=Brassica napus var. MENTOR; 3=Brassica napus var. AVATAR (no resistance); 4=Brassica rapa var. Granaat (no resistance); 5=Brassica napus NPZ-line (CRE1 resistance). A disease index below 0.25 indicates resistance of the tested plant line.

FIG. 4 shows the results of a clubroot infection with six Canadian isolates of Plasmodiophora brassicae on a Brassica differential set including new CR. Numbers on the x-axis represent different plant genotypes: 1=Brassica rapa var. Granaat (no resistance); 2=Brassica napus NPZ-line with QTL on A03; 3=Brassica napus (CRE1 resistance); 4=Brassica napus with QTL on A08; 5=new clubroot resistant line with QTL on chromosome A06. A disease index below indicates resistance of the tested plant line.

FIG. 5 shows the workflow for the generation of new CR in Brassica napus. Bn-1750 is Brassica napus crossing parent with partial resistance to Plasmodiophora brassicae and Br-14 is the source new CR from Brassica rapa.

FIG. 6 shows the results of an infection with Plasmodiophora brassicae isolate Pb #0 on a collection of Brassica napus lines at back-cross generations BC1F3 and BC2F3. All respective progenitor (BCxF2) plants were shown before to carry a homozygous allele indicating new CR. A, Numbers 1 to 15 are lines of BC1F3; B, numbers 16 to 37 are lines of BC2F3; 38=Brassica napus var. AVATAR (no resistance); 39=Brassica napus NPZ-line (CRE1 resistance); 40=Brassica rapa var. Granaat (no resistance).

FIG. 7 shows number of contigs per chromosome. Sequence reads specific for new CR were de novo assembled into contigs and aligned with reference genome Darmor-bzh 4.1.

FIG. 8 shows the distribution of new CR specific contigs from FIG. 7 along chromosome A06 of Darmor-bzh (version 4.1). Arrow indicates the physical position of molecular marker according to SEQ ID NO: 3 derived from the major QTL of the mapping population.

### DETAILED DESCRIPTION OF THE INVENTION

### Definitions

The present invention may be understood more readily by reference to the following detailed description of the preferred embodiments of the invention and the examples included herein. Unless otherwise noted, the terms used herein are to be understood according to conventional usage by those of ordinary skill in the relevant art.

It is to be understood that as used in the specification and in the claims, “a” or “an” can mean one or more, depending upon the context in which it is used. Therefore, reference to “a nucleic acid” can mean that at least one nucleic acid can be utilized.

It is to be understood that the term “comprising” is not limiting. Herein, the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(i)”, “(ii)”, “(iii)” etc. are used herein for distinguishing between similar elements and not necessarily for describing a sequential or chronological order. Such terms are interchangeable under appropriate circumstances and the embodiments of the invention described herein are capable of operation in other orders than the ones described herein.

The term “Brassica plant” refers to a plant belonging to the Brassicaceae family of flowering plants. Preferably, the term “Brassica plants” comprises the amphidiploid Brassica napus (AACC, 2n=38), Brassica juncea (AABB, 2n=36), Brassica carinata (BBCC, 2n=34), and the diploid Brassica rapa (syn. Brassica campestris) (AA, 2n=20), Brassica oleracea (CC, 2n=18) or Brassica nigra (BB, 2n=16).

The term “amphidiploid” describes an organism, cell, or nucleus that contains diploid sets of chromosomes originating from two different species.

Whenever reference to a “plant cell, plant or part thereof” or “seed” according to the invention is made, it is understood that plant parts (cells, tissues or organs, seed pods, seeds, severed parts such as roots, leaves, flowers, pollen, etc.), progeny or descendants of the plants which retain the distinguishing characteristics of the parents (especially the fruit dehiscence properties), such as seed obtained by selfing or crossing, e.g. hybrid seed (obtained by crossing two inbred parental lines), hybrid plants and plant parts derived therefrom are encompassed herein, unless otherwise indicated.

As used herein the term “progeny” or “descendent” is intended to mean the offspring or the first and all further descendants from a cross with a plant of the invention that shows the resistance trait and/or carries the genetic determinant underlying the trait. Progeny of the invention comprises descendants of any cross with a plant of the invention that carries the genetic determinant causing the resistance trait.

The term “F1 population” or “F1 generation” or “F1” as used herein refers to the first filial generation produced by a cross. The term “F2 population” or “F2 generation” or “F2” as used herein refers to offspring produced by self-pollination of individuals of an F1 generation.

The term “variety” or “cultivar” refers to a plant genotype that is officially approved for commercial production, which is distinct, stable and uniform in its characteristics when propagated.

The term “line” as used herein refers to a homozygous inbred line that has been generated by consecutive selfing.

The term “clubroot” as used herein refers to the disease caused by the pathogen Plasmodiophora brassicae, a common disease affecting plants of the Brassicaceae family that presents with symptoms including yellowing, wilting, stunted growth of the plant and the formation of galls on the plant's roots, which can eventually lead to death of the plant.

The term “clubroot resistance”, “CR” or “resistant against clubroot disease” as used herein refers to resistance to one or more Plasmodiophora brassicae isolates, such as, but not limited to, resistance to the Plasmodiophora brassicae used herein. Said resistance refers to a reduction in damage caused by clubroot infection compared to damage caused on control plants. Damage can be assessed as, for example, formation of club-shaped galls on the roots, occurrence of wilting, stunting, yellowing, premature senescence etc. In particular, a reduction in damage is manifested in a reduced yield loss when plants are grown under disease pressure in the field, compared to control plants. Such reduction in yield loss can, for example, be due to the fact that the infection, reproduction, spread or survival of the pathogen is reduced or prevented in plants with enhanced resistance. Said resistance may also refer to plants that are completely resistant, i.e., plants on which no disease symptoms are found.

Clubroot resistance can be assessed using a scale from zero to three, where 0=no galling, 1=a few small galls, 2=moderate galling, and 3=severe galling. Based on the scoring values the disease index (DI) for each plant line was calculated as follows:

$$DI = \frac{( no * 0 + n 1 * 1 + n 2 * 2 + n 3 * 3 )}{( N * 3 )} ,$$

where n0 to n3 is number of plants in the indicated class and N is the total number of plants tested.

It is understood that environmental conditions, such as location, weather conditions and disease pressure, as well as individual perception of the person assessing disease symptoms, can have an effect on the scoring of clubroot resistance. Hence, variation in these factors in comparative tests should be minimized. Any other resistance ratings known in the art can be applied in accordance with this invention to compare the plants of the invention with control plants.

As used herein, the term “quantitative trait locus” or “QTL” refers to a segment or region of DNA containing or linked to a gene or genes underlying a quantitative trait in the phenotype of a population of organisms. For the purposes of the present disclosure, the trait of particular interest is clubroot resistance. A quantitative trait is a trait that varies in degree and which can be attributed to polygenic effects, i.e. a product of two or more genes.

The term “locus” as used herein refers to a certain place or position on the genome, e.g. on a chromosome or chromosome arm, which comprises one or more genetic factors, for example one or several genes, contributing to a trait, such as a resistance to a disease.

The term “marker” as used herein, refers to a measurable, genetic characteristic with a fixed position in the genome, which is normally inherited in a Mendelian fashion, and which can be used for mapping of a trait of interest. Thus, a molecular marker may be a short DNA sequence, such as a sequence surrounding a single base-pair change, i.e. a single nucleotide polymorphism or SNP, or a long DNA sequence, such as microsatellites or Simple Sequence Repeats (SSRs). The nature of the marker is dependent on the molecular analysis used and can be detected at the DNA, RNA or protein level.

The term “single nucleotide polymorphism” or “SNP” refers to a DNA sequence variation occurring when a single nucleotide in the genome differs between members of a species or paired chromosomes in an individual. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed “synonymous” (sometimes referred to a silent mutation). If a different polypeptide sequence is produced, they are termed “non-synonymous”. A non-synonymous change may either be missense or nonsense, where a missense change results in a different amino acid and a nonsense change results in a premature stop codon. SNPs that are not in protein-coding regions may still have consequences for e.g. gene splicing, transcription factor binding, or the sequence of non-coding RNA (e.g. affecting transcript stability, translation). SNPs are usually biallelic and thus easily assayed in plants and animals.

The term “allele(s)”, such as of a gene, means any of one or more alternative forms of a gene at a particular locus. In a diploid cell of an organism, alleles of a given gene are located at a specific location or locus on a chromosome. Typically, one allele is present on each chromosome of a pair of homologous chromosomes, although this does not have to be the case for transgenic alleles.

The term “monomorph” as used herein describes the situation when a bi-allelic marker detects only one of two possible alleles in a given plant population. The lack of nucleotide variation for this position precludes linkage between a marker specific for this nucleotide and any trait that segregates in this population.

The phrase “detecting a QTL” or “detecting the presence of the QTL” as used herein refers to establishing the presence of a nucleic acid sequence associated with the QTL using appropriate methods. A preferred method of detecting the presence of the QTL in the genomic DNA is by polymerase chain reaction (PCR).

The terms “genetically linked”, “linked”, “linked to” or “linkage”, as used herein, refers to a measurable probability that genes or markers located on a given chromosome are being passed on together to individuals in the next generation. Thus, the term “linked” may refer to one or more genes or markers that are passed together with a gene with a probability greater than 0.5 (which is expected from independent assortment where markers/genes are located on different chromosomes). Because the proximity of two genes or markers on a chromosome is directly related to the probability that the genes or markers will be passed together to individuals in the next generation, the term genetically linked may also refer herein to one or more genes or markers that are located within about 50 centimorgan (cM) or less of one another on the same chromosome.

The term “centimorgan” or “cM” refers to a unit of recombination frequency for measuring genetic linkage, defined as the distance between genes or markers for which one product of meiosis in 100 is recombinant, or in other words, the centimorgan is equal to a 1% chance that a marker at one genetic locus on a chromosome will be separated from a marker at a second locus due to crossing over in a single generation. It is often used to infer distance along a chromosome. The number of base pairs to which cM correspond varies widely across the genome (different regions of a chromosome have different propensities towards crossover) and the species (i.e. the total size of the genome).

The term “a marker linked to a QTL” as used herein refers to a marker in a region in the genome that inherits with the QTL or CR locus as a single genetic unit in more than 50% of the cases.

The term “interval marker” or “flanking marker” as used herein refers to a marker that defines one of the termini of an interval (and is included in that interval). It will be clear that any of such intervals may comprise further markers. Two or more interval markers may be located at the respective termini of the region of interest (e.g. a QTL). For example, one interval marker may be located at the 5′ end of the QTL and one interval marker may be located at the 3′ end of the QTL. By detecting the presence of both markers, it can be ensured that the entire QTL is present in the analyzed sequence.

The term “interval” refers to a continuous linear span of chromosomal DNA with termini defined by map position and/or markers. A QTL positioned “between” two markers or “within an interval” is a QTL within a continuous linear span of chromosomal DNA that is flanked by two markers.

The term “LOD score” or “logarithm of odds score” refers to a statistical measurement of QTL linkage. It represents the log to the base 10 of the ratio of probability of obtaining the given data assuming linkage between the two genes with a specified frequency of recombination to the probability of getting the same data with independent segregation. The LOD score compares the likelihood of obtaining the test data if the two loci are indeed linked, to the likelihood of observing the same data purely by chance. Positive LOD scores favor the presence of linkage, whereas negative LOD scores indicate that linkage is less likely. A LOD score of 1 signifies that linkage with the given frequency of recombination is 10 times more likely than independent segregation, LOD value of 2 and, 3 will reveal the linkage to be 100 and 1000 times, respectively, more likely than independent assortment.

The term “genomic region” as used herein describes a continuous span of chromosomal DNA which can be defined by its location within an individual genome relative to a reference genome and may for example comprise a QTL, one or more genes, one or more intergenetic regions, one or more markers or one or more SNPs.

The term “genomic region corresponding to” as used herein refers a genomic region within an individual genome that corresponds to a genomic region within a reference genome, which is defined by a specific position within the reference genome or by a specific position within the genome map of the reference genome.

The term “reference genome” as used herein refers to a digital nucleic acid sequence database assembled from nucleic acid sequencing of one or more individuals of the same species, variety or cultivar. The sequencing reads are assembled into individual, usually continuous, sequences representing the different chromosomes of the whole genome of the species, variety and cultivar or line. The reference genome serves as a representative example of the genome sequence of this species, variety or cultivar or line. The reference genome is annotated with continuous base pair count and mapping of genetic elements such as for example known SNPs, coding elements (e.g. genes), non-coding elements or epigenetic marks.

The “Darmor reference genome” as used herein refers to the assembled reference genome of the Brassica napus line Darmor-bzh, version Darmor-bzh v.4.1. This sequence is available at http://www.genoscope.cns.fribrassicanapus/Map positions in the reference genome refer to nucleotides of the respective chromosomes.

The term “contig” as used herein refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequence and to overlapping physical segments (fragments) contained in clones depending on the context.

The term “scaffold” as, used herein, refers to overlapping DNA contigs that together represent a consensus region of DNA.

The phrase “obtaining a sample” as used herein refers to the selection and removal of one or several plant parts such as leaf, stem, root, fruit, flower or seed from a plant, or removal of an appropriate number of plant cells from a plant or a cell culture.

The phrase “isolating genomic DNA” as used herein refers to extraction of the genomic DNA from the cell nucleus of cells obtained from one or several organisms. Genomic DNA isolation or extraction from plant cells comprises the steps of breaking down the cell wall and disrupting the plasma membrane (lysis) and the nucleus, by using chemical (e.g. enzymes, lysozyme, proteinase) or mechanical (bead beating, freeze-thawing, grinding) methods. The genomic DNA is then purified using a method comprising centrifugation, filtration, column purification or phenol-chloroform isolation of the DNA. This is preferably achieved using commercial DNA isolation and/or DNA purification kits.

The term “PCR” or “polymerase chain reaction” as used herein refers to a method of amplifying a specific region of DNA for further analysis or processing by for example DNA sequencing or visualization. The method comprises the use of short oligonucleotides (primers), a DNA-polymerase, deoxynucleoside triphosphate, a template DNA and a thermal cycler, the method comprising several cycles of denaturation, primer hybridization or annealing and elongation.

The term “primer” as used herein refers to a short synthetic oligonucleotide molecule that anneals to a sequence of interest in a DNA complementary to its own sequence at a specific annealing temperature. Preferably, the annealing sequence of the primer is 15 to 25 nucleotides in length and comprises a unique sequence in the target DNA. A primer may also comprise additional sequences and tags such as fluorescent labels.

The term “hybridization” as used herein refers to a process in which single-stranded DNA or RNA molecules anneal to complementary DNA or RNA. Hybridization occurs during DNA replication and transcription of DNA into RNA, Southern blot, Northern blot, PCR, DNA sequencing, DNA-DNA hybridization or Fluorescence in situ hybridization or RNA in situ hybridization.

The term “KASP assay” as used herein describes an assay for detection of SNP markers. KASP genotyping assays are based on competitive allele-specific PCR and enable biallelic scoring of single nucleotide polymorphisms (SNPs) and insertions and deletions (Indels) at specific loci. For developing the KASP-assay 70-100 base pairs upstream and 70-100 base pairs downstream of the SNP are selected and two allele-specific forward primers and one allele specific reverse primer is designed. See e.g. Allen et al. 2011, Plant Biotechnology J. 9, 1086-1099, especially p 1097-1098 for KASP assay method.

The term “SNP analysis” as used herein refers to the detection of one or more specific SNPs within the DNA extracted from a sample from an individual organism or a population of organisms using known methods. The analysis typically includes comparison between individuals or populations or comparison with a reference sequence.

The term “genotyping” as used herein refers to the process of determining differences in an individual genome by revealing the alleles or SNPs that an individual has inherited from their parents using known methods.

The term “nucleic acid sequencing” refers to the process of determining the nucleotide sequence of a given DNA fragment. Common nucleic acid sequencing methods comprise next generation sequencing and Sanger sequencing.

The term “next-generation sequencing” or “NGS” refers to the high-throughput DNA sequencing using massively parallel sequencing of spatially separated, clonally amplified DNA templates or single DNA molecules using methods such as pyrosequencing or Illumina dye sequencing.

The term “Sanger sequencing”, also called “first-generation sequencing”, refers to a method of DNA sequencing based on the electrophoretic separation of chain-termination products produced in individual sequencing reactions.

The term “nuclease based detection” refers to a method of detection of DNA mismatches between two DNA fragments that differ in one nucleotide using a nuclease, e.g. Surveyor nuclease, that specifically detects mismatches in the DNA and then cleaves the DNA at the site of the mismatch.

The term “antibody” refers to an immunoglobulin or a fragment thereof, which is a protein produced by the immune system of mammals that recognizes and binds a unique antigen. The antigen is a molecule or molecular structure that can be a protein or part of a protein or other macromolecule.

The term “nucleic acid probes” or “hybridization probe” refers to a fragment of DNA or RNA of variable length which can be radioactively or fluorescently labelled or comprise a sequence that can be recognized by another probe or by an antibody (a “tag”). It can be used in samples that contain DNA or RNA to detect the presence of a nucleic acid sequence that is complementary to the sequence of the probe.

The term “DNA chip technology”, “DNA microchip”, “biochip” or “DNA microarray” refers to a collection of microscopic DNA molecules attached to a solid surface or beads. DNA chip technology can for example be used for SNP detection, gene expression profiling or chromatin immunoprecipitation.

The term “zygosity status” as used herein refers to the type and number of allele present at a specific locus in the genome on a pair of homologous chromosomes. If both alleles of a diploid organism are the same, the organism is homozygous at that locus. If they are different, the organism is heterozygous at that locus. If one allele is missing, it is hemizygous, and, if both alleles are missing, it is nullizygous.

The term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent. In tetraploid species, two sets of diploid genomes exist, whereby the chromosomes of the two genomes are referred to as “homoeologous chromosomes”.

The term “dominant” as used herein refers to an allele or SNP that determines the phenotype when present in the heterozygous or homozygous state, masking or overriding the effect of a different variant of the same gene on another chromosome. The term “recessive” refers to an allele or SNP that only determines the phenotype when present in the homozygous state.

The terms “nucleic acid” or “nucleic acid molecule” or “nucleic acid sequence” or “nucleotide sequence” are used interchangeably herein to refer to a biomolecule composed of nucleotides. The nucleic acid molecule can be comprised within an eukaryotic or prokaryotic organism, a eukaryotic or prokaryotic cell, a cell nucleus or a cell organelle, as part of a genome or as an individual molecule; or it can be comprised within a plasmid, a vector, an artificial chromosome; a nucleic acid can also exist outside of a cell, in vesicles, viruses or freely circulating, i.e. in blood; it can be isolated in a suitable composition, in a fixed or frozen tissue, or dried. The nucleic acid can be synthesized or naturally occurring, i.e. isolated from nature.

The terms “sequence Identity”, “% sequence identity”, “% identity”, “% identical” or “sequence alignment” are used interchangeably herein and refer to the comparison of a first nucleic acid sequence to a second nucleic acid sequence, or a comparison of a first amino acid sequence to a second amino acid sequence and is calculated as a percentage based on the comparison. The result of this calculation can be described as “percent identical” or “percent ID.” A sequence identity may be determined by a program, which produces an alignment, and calculates identity counting both mismatches at a single position and gaps at a single position as non-identical positions in final sequence identity calculation. The sequence identity is determined over the entire length of the first and second nucleic acid sequence.

The term “open reading frame” or “ORF” as used herein refers to a continuous stretch of DNA codons between a translation start and a translation stop. The length of an ORF is a multiple of 3 because DNA codons consist of three nucleotides. An ORF typically comprises exons and may be interspersed by introns or non-coding DNA, and it may be present within genomic DNA, it may be part of a messenger RNA (mRNA) or it may be present in an artificial expression system such as a plasmid vector or artificial chromosome. An ORF is “encoding a protein”, since the three-nucleotide codons present in the ORF can be translated into a protein sequence comprising amino acids by the translation machinery of a cell.

The term “encoded protein” refers a protein that consists of a chain of amino acids, which results from a sequence that is encoded by a nucleic acid molecule comprising three-nucleotide codons.

The term “conferring resistance” or “confers resistance” as used herein refers to a gene, nucleic acid, allele, marker, SNP, or coding variant resulting in the expression of one or more new or changed proteins, in a change of the expression or secretion levels of one or more proteins or in a functional change to one or more proteins, e.g. a changed binding or interaction activity, which leads to an increased resistance to a disease or pathogen.

The term “transferring resistance” as used herein refers to the process of passing on a resistance to a disease, such as a resistance to clubroot disease, from a parent organism to a descendant organism through crossing or breeding. The term may also refer to transferring resistance through other methods such as transgenesis or genetic transformation.

The term “introgression” as used herein refers to the incorporation of genetic material, such as alleles or genes, from one species into the gene pool of a second, divergent species. Introgression commonly involves hybridization and backcrossing.

The term “hybridization” as used herein refers to the cross between two true-breeding organisms, which results in an F1 hybrid generation that carries one allele from each parent.

The term “breeding” or “plant breeding” as used herein refers to the purposeful manipulation of plant species in order to create desired genotypes and phenotypes with specific traits, this manipulation involving controlled pollination and/or genetic engineering and artificial selection of progeny.

As used herein, the term “conventional breeding” refers to the selection and propagation of plants with desirable characteristics and the elimination of those with less desirable characteristics.

The term “crossing” as used herein refers to the interbreeding of two individual plants in order to produce new plant varieties that comprise specific genes or traits of both parents or in order to introduce specific genes or traits into a new genetic background.

The term “self-pollination” or “selfing” refers to the natural or artificial self-fertilization of an individual plant, which leads to a loss of genetic variation and keeps desired traits stable within a population.

The term “doubling haploidy” refers to the process of producing homozygous double haploid plants. This is achieved by inducing haploid embryos to undergo artificial chromosome doubling with the use of a meiosis blocking agent to form doubled haploids. Haploid embryos can be produced using known in vivo or in vitro methods.

The term “single seed descent” refers to a breeding method whereby individuals of a plant population are propagated over various generations by using only a single seed derived from the respective progenitor.

The term “pedigree breeding” refers to a breeding method whereby a pure line is obtained through many generations of controlled breeding through self-pollination for desirable traits. In other examples, selection of individuals is based on their pedigree information to generate segregating populations. Members of this population undergo two or three cycles of phenotypic evaluation and selfing until sufficiently homozygous lines with the desired traits are achieved.

The term “backcrossing” refers to the crossing of a hybrid plant with one of its parents or an individual genetically similar to its parent, in order to achieve offspring with a genetic identity which is closer to that of the parent or in order to transfer a desirable trait in an individual of inferior genetic background into a preferable genetic background.

The term “hybrid breeding” refers to the crossing of two homozygous, genetically different parental lines. The heterozygous offspring (“hybrid”) often presents with enhanced traits such as greater biomass and fertility, this effect is called “hybrid vigor” or “heterosis”.

The term “somatic hybridization” or “protoplast fusion” refers to a type of genetic modification by which the protoplasts of cells from two distinct plant species are fused together to form a new hybrid plant. The hybrid offspring carries characteristics of both parents. This technique can be used to cross plants that cannot reproduce sexually or are sterile.

The phrase “breeding by genetic transformation” as used herein refers to the production of a plant with a desired trait or traits by introducing a specific functional nucleic acid molecule, e.g. a gene or a small interfering RNA, thereby producing a transgenic plant. The term “transformation” as used herein refers to the genetic modification of a cell by incorporation of genetic material from the outside. Plant transformation may make use of vectors (e.g. Agrobacterium or virus).

The term “introducing” as used herein includes stable integration by means of transformation including Agrobacterium-mediated transformation, transfection, microinjection, biolistic bombardment, insertion using gene editing technology like CRISPR systems (e.g. CRISPR/Cas, in particular CRISPR/Cas9 or CRISPR/Cpf1, CRISPR/CasX, CRISPR/CasY, CRISPR/Csm1 or CRISPR/MAD7), TALENs, zinc finger nucleases or meganucleases, homologous recombination optionally by means of one of the mentioned gene editing technologies including preferably a repair template, modification of endogenous gene using random or targeted mutagenesis like TILLING or below mentioned gene editing technologies, etc. The term “introducing” may or may not encompass the introgression using conventional or non-conventional breeding techniques.

The terms “transgene” as used herein refers to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. The term “transgenic” refers to an organism carrying a transgene, and the term “transgenesis” refers to the processes or methods of producing a transgenic organism.

The term “genome editing” as used herein refers to strategies and techniques for the targeted, specific modification of any genetic information or genome by means of or involving a double-stranded DNA break inducing enzyme or single-stranded DNA or RNA break inducing enzyme.

As such, the terms comprise gene editing, but also the editing of regions other than gene encoding regions of a genome, such as intronic sequences, non-coding RNAs, miRNAs, sequences of regulatory elements like promoter, terminator, transcription activator binding sites, cis or trans acting elements. Additionally, the terms may comprise base editing for targeted replacement of single nucleobases. It can further comprise the editing of the nuclear genome as well as other genetic information, i.e. mitochondrial genome, chloroplast genome or an artificial genome or chromosome as well as miRNA, pre-mRNA or mRNA.

The term “targeted mutagenesis” or “site-directed mutagenesis” as used herein refers to a method of making specific and intentional changes to a DNA sequence using methods of molecular biology. These methods may include genome editing, PCR-based site-directed mutagenesis, overhang or overlap PCR cloning methods, or de novo nucleic acid molecule synthesis.

The term “non-targeted mutagenesis” or “random mutagenesis” refers to a method wherein a mutagen or molecular biology method is used to introduce random changes into DNA. This can be achieved, for example, using UV radiation, mutagenic chemicals such as ethyl methanesulfonate (EMS) or nitrous acid, error prone PCR, DNA shuffling, insertion mutagenesis kits, or mutator strains with impaired DNA repair machinery.

The term “harvested part” as used herein refers to any picked or collected part of a crop plant.

The term “feed product” refers to any agricultural foodstuff made from plant material used to feed domesticated livestock, also called “fodder” or “provender”. Fodder includes for example hay, straw, silage, compressed and pelleted feeds, oils, sprouted grains or legumes.

The term “crushed Brassica seed” as used herein refers, e.g, to the of seed from a Brassica plant that has been industrially processed in the first step of the production of oil, meal and other by-products.

The term “oil” as used herein refers to an edible vegetable oil that is a product obtained from a Brassica seed that can be used as a food product or in the production of biofuels. Oil from Brassica napus is called rapeseed oil. Oil from Brassica napus cultivars with low levels of erucic acid is also called canola oil.

The term “meal” as used herein refers to a product of Brassica seed processing that is obtained after crushing of the seed and removal of the oil.

The term “derived products” as used herein refers to any final product or by-product obtained from Brassica seed crushing other than oil and meal.

### Further Embodiments

At present, rapeseed cultivars with resistance to clubroot disease are usually derived from the variety ‘MENDEL’ and thus inherited a limited set of resistance alleles outside the A06 chromosome. As the resistance conferred by these alleles is only partial, various Plasmodiphora brassicae isolates can still infect these plants. Brassica rapa line Br-14 carries additional alleles conferring resistance against clubroot disease. Hence, the QTL described herein provides improved and increased resistance compared to known clubroot resistance alleles.

In one embodiment, the invention provides a method for identifying or selecting a Brassica plant cell, Brassica plant or part thereof, or a Brassica seed comprising a QTL that confers increased resistance against clubroot disease compared to clubroot resistance derived from a Brassica variety selected from the group consisting of Brassica napus variety MENTOR, Brassica napus variety AVATAR, Brassica rapa variety Granaat, and Brassica napus variety MENDEL.

In one embodiment, the invention provides a method for identifying or selecting a Brassica plant cell, Brassica plant or part thereof, or a Brassica seed comprising a QTL that confers increased resistance against clubroot disease compared to clubroot resistance derived from the variety MENDEL.

In another embodiment, the invention provides a method for identifying or selecting a Brassica plant cell, Brassica plant or part thereof, or a Brassica seed comprising a QTL on chromosome A06 that confers increased resistance against clubroot disease compared to a Brassica plant cell, Brassica plant or part thereof, or a Brassica seed comprising a QTL on a chromosome other than chromosome A06.

In another embodiment, the invention provides a method for identifying or selecting a Brassica plant cell, Brassica plant or part thereof, or a Brassica seed comprising a QTL on chromosome A06 that confers increased resistance against clubroot disease compared to a Brassica plant cell, Brassica plant or part thereof, or a Brassica seed comprising a QTL on chromosome A08 or chromosome A03.

The present inventors identified Plasmodiphora brassicae isolate Pb #0, which infects rapeseed carrying so far known CR alleles of Brassica napus. Yet, this isolate does not cause clubroot disease in Brassica rapa line Br-14.

The present invention is based on the identification of a new QTL that confers resistance against clubroot disease in Brassica rapa Br-14. The inventors further achieved transferring this QTL from Brassica rapa Br-14 to Brassica napus by introgression. The inventors were able to identify introgression of the QTL from Brassica rapa Br-14 into Brassica napus by crossing Brassica rapa Br-14 with Brassica napus and screening the resulting F1 generation for resistance against Plasmodiphora brassicae isolate Pb #0. Screening the F2 generation for resistance against Pb #0 revealed that the resistance segregates according to the Mendelian law. Accordingly, the inventors concluded that the resistance introgressed into the Brassica napus genome and it involves a single allele.

In addition to the mapping of the QTL conferring clubroot resistance introgressed into Brassica napus, the inventors were also able to identify the QTL conferring clubroot resistance in the genome of Brassica rapa Br-14.

Hence, in one embodiment, the invention provides a method for identifying or selecting a Brassica plant cell, Brassica plant or part thereof, or a Brassica seed comprising a QTL that confers resistance against clubroot disease, the method comprising detecting a QTL in the genome of the Brassica plant cell, Brassica plant or part thereof, or Brassica seed localizing within a genomic region, wherein the genomic region corresponds to position 4392950 bp to 9649373 bp of chromosome A06 of the B. rapa v3.0 genome (Zhang et al., 2018).

The present invention thus provides a Brassica plant resistant to clubroot disease. It further provides methods that allow detecting whether or not a plant is resistant against clubroot disease.

In one embodiment, a plant resistant to clubroot disease exhibits a score of 0 on the scale of to 3. In one embodiment, a plant resistant to clubroot disease exhibits a score of below 1 on the scale of 0 to 3. In one embodiment, a plant resistant to clubroot disease exhibits a score of below 2 on the scale of 0 to 3.

In one embodiment, a plant resistant to clubroot disease is a plant in which the percentage of the root system which is clubbed is 0%, or less than 5%, or less than 10%, or less than 15%, or less than 20%, or less than 25%, or less than 30%, or less than 35%, or less than 40%, or less than 45%, or less than 50%, or less than 55%, or less than 60%, or less than 65%, or less than 70%, or less than 75%, or less than 80%, or less than 85%, or less than 90%, or less than 95%, or less than 100%.

In one embodiment, a plant resistant to clubroot disease exhibits a disease index (DI) of less than 1.0, or less than 0.95, or less than 0.9, or less than 0.85, or less than 0.8, or less than or less than 0.7, or less than 0.65, or less than 0.6, or less than 0.55, or less than 0.5, or less than 0.45, or less than 0.4, or less than 0.35, or less than 0.3, or less than 0.25, or less than 0.2, or less than 0.15, or less than 0.1, or less than 0.05. In a preferred embodiment, a plant resistant to clubroot disease exhibits a disease index of less than 0.1.

In one embodiment, a plant with increased resistance to clubroot disease is a plant exhibiting less clubbing compared to a plant without increased resistance to clubroot disease. In a preferred embodiment, a plant with increased resistance to clubroot disease is a plant exhibiting a decrease of clubbing by at least 30%, at least 50% or at least 80% compared to a plant without increased resistance to clubroot disease.

In one embodiment, a plant with increased resistance to clubroot disease exhibits a disease index (DI) that is lower than the DI of a plant without increased resistance to clubroot disease. In one embodiment, the DI of a plant with increased resistance to clubroot disease is reduced by at least 0.3, at least 0.5 or at least 0.8 compared to the DI of a plant without increased resistance to clubroot disease.

In one embodiment, the plant with increased resistance to clubroot disease comprises a nucleic acid of the invention and the plant without increased resistance to clubroot disease does not comprise a nucleic acid of the invention. In one embodiment, the plant with increased resistance to clubroot disease comprises a QTL of the invention and the plant without increased resistance to clubroot disease does not comprise a QTL of the invention.

In one embodiment, the plant is a Brassica species. In a preferred embodiment, the plant is Brassica napus.

The above described embodiments equally apply to a population of pants.

Seeds of Brassica napus that comprise the nucleic acid of the invention on chromosome A06 which confers the resistance against Plasmodiophora brassicae as described herein were deposited with NCIMB Ltd, Ferguson Building, Craibstone Estate, Bucksburn, Aberdeen AB21 9YA, UK on Dec. 3, 2020 under deposit accession number NCIMB 43700.

### Examples

To accomplish CR to a broad spectrum of pathotypes in Brassica napus a strategy with three consecutive steps was used:
(1) The inventors aimed at increasing the likelihood of generating full constitution of Br-14 resistance in Brassica napus through the transfer of so far unknown resistance traits of Br-14. Therefore, the inventors crossed resistance donor line Brassica rapa Br-14 with the Brassica napus breeding line Bn-1750. The latter possess a partial resistance inherited from Br-14 and represents the available CR in rapeseed today.
(2) The inventors used a pathotype of Plasmodiophora brassicae that is virulent on available resistance inside Brassica napus but not on Br-14. This highly virulent Plasmodiophora brassicae isolate (Pb #0) was taken from a field in Germany and represents pathotypes able to induce disease on the recipient Brassica napus parent Bn-1750. Consequently, any offspring from a cross between Bn-1750 and Br-14 showing no disease symptoms would reveal a new CR (CR).
(3) As interspecies crosses (e.g. Brassica rapa x Brassica napus) often result in aneuploid karyotypes (additional chromosomes) that do not segregate according to Mendelian law and thus are not suitable for variety breeding, a pre-requisite for success of the whole strategy was that progenies of the crosses with the new CR must segregate in a F2 population as it would be expected from any kind of Brassica rapa introgression into the Brassica napus genome. However, because of the broad spectrum of Plasmodiophora brassicae pathotypes Br-14 is resistant to, it was neither clear how many genomic loci would be involved nor whether the inheritance is dominant or recessive.

Plasmodiophora brassicae Material

Plasmodiophora brassicae isolates were extracted from diseased rapeseed plants (roots with clubs) taken from different field sites in Europe. Propagation and characterization of the isolates is done under greenhouse conditions. For storage, infected roots are air-dried or kept at −18° C. The Plasmodiophora brassicae isolate used in the present file was taken from a field in Germany and is named Pb #0. This isolate can infect all currently known resistances in Brassica napus but not the Brassica rapa resistance of Br-14 donor line used here.

Preparation of Plasmodiophora brassicae Inoculum

Plasmodiophora brassicae inoculum was prepared by homogenizing 3-4 clubbed root samples from greenhouse infection using a blender in approximately 400 ml water and filtering of the homogenate through a gaze with a mesh diameter of 5 μm. Resting spore concentration of the filtrate was estimated with a haemocytometer and adjusted to 10⁶ spores/ml.

Plant Material

The source of CR was a Brassica rapa fodder turnip of high resistance complexity (Br-14), meaning that none of 60 P. brassicae isolates tested (see “Infection with Plasmodiophora brassicae”) was able to infect this line under greenhouse conditions. As Brassica napus crossing parent we chose a spring-type rapeseed line from NPZ (Bn-1750). Bn-1750 contains part of the CR of the Brassica rapa resistance donor line used in this invention. The resistance of Bn-1750 shares the race-specificity of CRE1. In the infection tests the following lines or varieties were taken alongside as references: Brassica rapa variety Granaat (susceptible), rapeseed variety AVATAR (susceptible), rapeseed variety MENDEL (race-specific resistance), Brassica napus CRE1 (extended race-specific resistance).

Infection with Plasmodiophora brassicae

A collection of Plasmodiophora brassicae isolates was extracted from diseased rapeseed plants (roots with clubs) in recent years taken from different field sites in Europe and Canada. The isolates constitute of various pathotypes.

For the preparation of Plasmodiophora brassicae inoculum 3 to 4 clubs per isolate were homogenized with a blender in ca. 400 ml water. The homogenate was filtered through a gaze with a mesh diameter of 5 μm. Spore concentration of the resulting filtrate was counted with a haemocytometer and adjusted to 106 spores/ml with water.

To grow the plants, seeds were first incubated on moistened paper in a Petri dish under fluorescent light. At 5 days after sowing 5 seedlings were transferred to a 9*9*9 cm plastic pot filled with potting soil, peat, and quartz sand (0.6:0.2:0.2). The pH of this substrate is typically between pH 5.5 and pH 6. Per genotype 3 pots were prepared. One day after transfer, seedlings were inoculated with a 4 ml spore suspension per plant by injecting spore suspension into the soil near the root zone. Pots were placed in a greenhouse at 22° C. and 18° C. day and night, respectively, with a 16-h photoperiod. Plants were kept under conditions of high soil humidity for the first two weeks. Six weeks after sowing, roots have been washed with tap water and examined for gall formation. The scoring of symptom development followed a scale of 0 to 3 classes, where 0=no galling, 1=a few small galls, 2=moderate galling, and 3=severe galling. Based on the scoring values (disease score) we calculated the disease index (DI) for each plant line:

DI=((n0*0+n1*1+n2*2+n3*3))/((N*3)).

Here, n0 to n3 is number of plants in the indicated class and N is the total number of plants tested. The DI can range between 0 and 1. A successful infection will give DI values of >0.8 for the susceptible reference. The reaction of the plant host to each field isolate is recorded as resistant if DI 0.25 or susceptible if DI>0.25.

The Plasmodiophora brassicae isolate used in the present file was taken from a field in Germany and is named Pb #0. This isolate can infect all currently known resistances in Brassica napus but not the Brassica rapa resistance of Br-14 donor line used here.

Crossing of Brassica napus with Brassica rapa Br-14

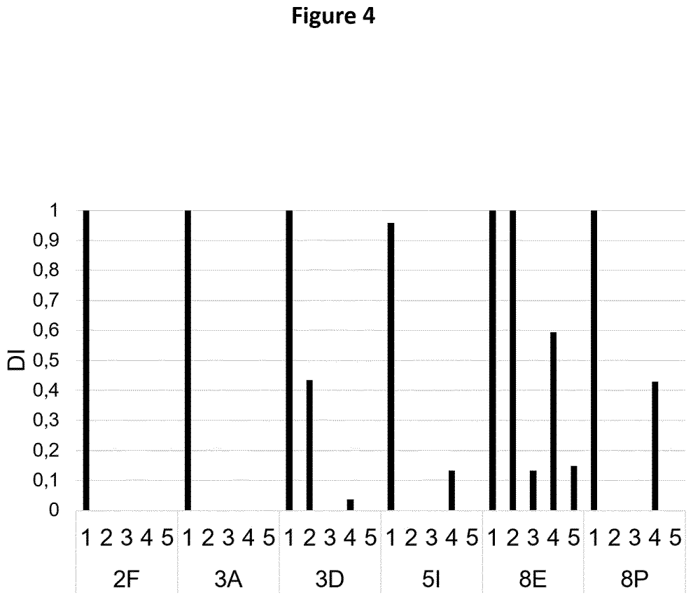

We performed crosses between spring-type oilseed rape (Bn-1750) carrying partial CR and the resistance donor line Brassica rapa Br-14 by hand pollination (FIG. 4). Resulting seeds of F1 generation were sown and infected with the Plasmodiophora brassicae isolate Pb #0 virulent on Bn-1750 under greenhouse conditions. Out of ten seeds 9 plants grew up and 8 turned out to be resistant. The latter have been selected for F2 seed production. The amount of harvested F2-seeds ranged from 20 seeds to 0.57 g per resistant plant. Seeds of the eight F2 harvests were sown as individual populations and number of developing seedlings ranged from 5 to 104 plants per population (Table 1). Subsequently, all F2 populations were infected with the Plasmodiophora brassicae isolate Pb #0 used before to distinguish resistant from susceptible plants.

TABLE 1
Clubroot infection of eight F2 populations from a cross between spring-type oilseed
rape (SOR, Bn-1750) and Brassica rapa BR-14 six weeks after infection with a
Plasmodiophora brassicae isolate Pb#0. Disease symptoms were scored on a scale of 0 (=
fully resistant) to 3 (= heavy gall formation). N = total number of germinated/infected plants.

| | | | | Score | Score | Score | Score | | |
|---|---|---|---|---|---|---|---|---|---|
| Nr. | Mother | Father | Generation | 0 | 1 | 2 | 3 | N | % resistant |
| 1 | SOR | Br-14 | F2 | 11 | 4 | 1 | 5 | 21 | 52, 38 |
| 2 | SOR | Br-14 | F2 | 16 | 3 | 0 | 3 | 22 | 72, 73 |
| 3 | SOR | Br-14 | F2 | 2 | 0 | 1 | 2 | 5 | 40, 00 |
| 4 | SOR | Br-14 | F2 | 78 | 6 | 10 | 10 | 104 | 75, 00 |
| 5 | SOR | Br-14 | F2 | 17 | 0 | 0 | 6 | 23 | 73, 91 |
| 6 | SOR | Br-14 | F2 | 14 | 1 | 1 | 9 | 25 | 56, 00 |
| 7 | SOR | Br-14 | F2 | 16 | 2 | 2 | 4 | 24 | 66, 67 |
| 8 | SOR | Br-14 | F2 | 25 | 1 | 1 | 4 | 31 | 80, 65 |

The number of resistant plants ranged from 40% to 80.65% among the F2-families. Surprisingly, we observed three populations with a proportion of resistant individuals close to or exactly 75%. This segregation ratio in a F2-generation indicates a stable introgression of a dominant gene at only one locus conferring a dominant resistance. Because of its segregation ratio for resistance and population size we choose F2 population Nr. 4 for further analysis. After disease scoring all resistant individuals were continued to grow for seed setting (F3 generation) and 18 resistant F2 plants were chosen for back-cross with Bn-1750 (=BC1).

Genotyping and Mapping of new CR in the genome of Brassica napus

Leaf samples of 104 individual plants were taken from a F2 population segregating for CR and analysed with a 15k Illumina Infinium SNP-chip at TraitGenetics (http://www.traitgenetics.com, Germany). Genotypic and phenotypic data were combined by simple interval mapping to calculate quantitative trait loci (QTL) using R-QTL (Bromann et al., 2003). For routine and high-throughput analysis single SNP derived from QTL-regions were translated into bi-allelic molecular marker using KASP-technology (https://www.lgcgroup.com, UK).

Genetic Characterization of New CR

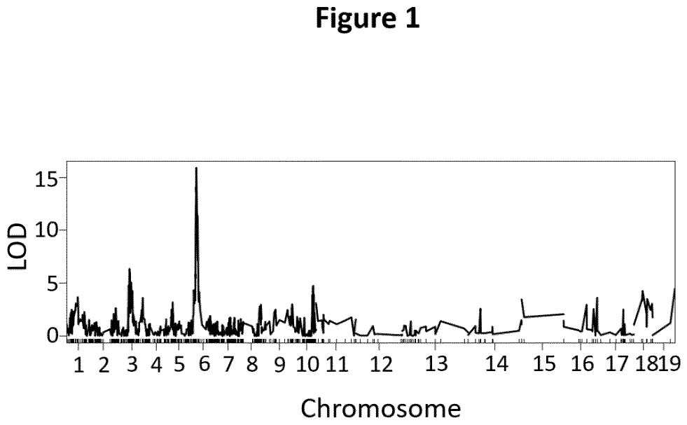

To position the new CR in the genome we took leaf samples of plants from F2 population Nr. 4 and performed SNP genotyping using a Brassica napus 15K Illumina Infinium DNA-Chip (Traitgenetics). The SNP-chip data were filtered for polymorphic marker and combined with phenotypic data to perform a QTL-analysis (R-QTL software, FIG. 1).

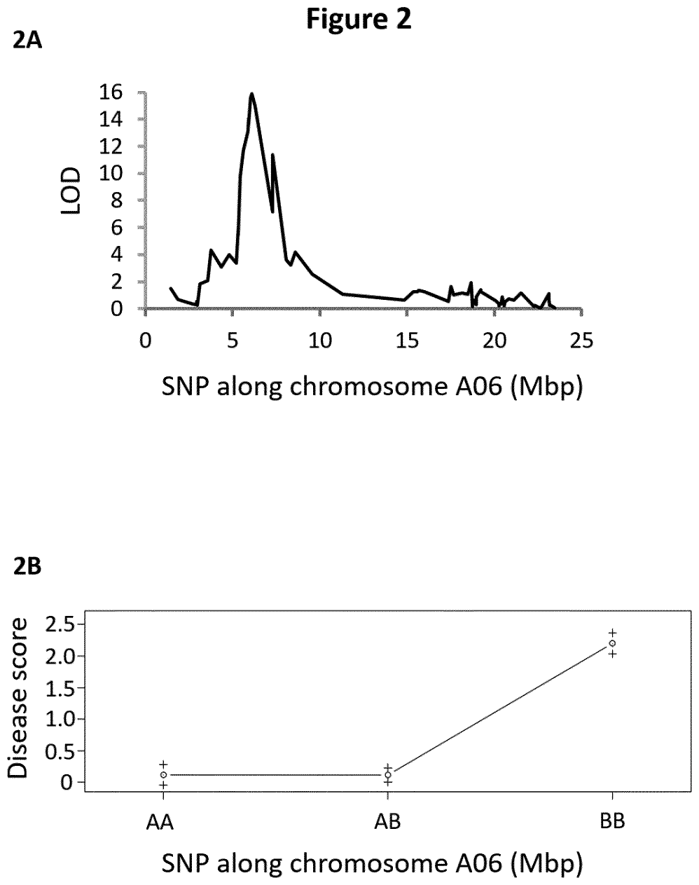

The QTL-mapping revealed one major QTL significantly linked to the new CR on chromosome A06 with a maximum LOD value of 15.9. A close-up view on chromosome A06 shows the position of the resistance QTL at ca. 6 Mbp according to Darmor-bzh reference genome (FIG. 2A). The most significant marker in the A06 QTL accounts for a decrease of 2 scoring notes (disease scores) and thus explains most of the observed resistance variance (FIG. 2B). Since the heterozygous genotype of this marker was enough to confer full resistance, a dominant inheritance was then inferred.

A SNP representative for the QTL was translated into a KASP-assay for routine molecular marker analysis (Tab. 2).

TABLE 2
List of markers inside the QTL on A06

| SEQ ID | | |
|---|---|---|
| NO: | Sequence in 5′-3′; last nucleotide specific for Br-14 | Comment |
| 1 | GTTATCTAGGAATAGAGCTTGAATAAATGATAGCCATCTT | interval marker |
| | GTGATGAACTT | |
| 2 | TTCTACGGGTGTGATAAATAGATATCTAAGGTAGAGTCCG | interval marker |
| | TGAGTTTCATC | |
| 3 | TGAACCCTGATTGTTCTTCACTTCAAATATCACACAGAAG | linked marker |
| | ATTGTATGCAG | |
| 4 | GTTTGTGTTTTGTGAATCTTAAATGCTTAACTAAGGAAAGT | linked marker |
| | TACTAAAAAG | |
| 5 | CGCGTGGCGACCATCTTAATTAACCGGTTTATCATCCAAT | linked marker |
| | CGATAAGTTTC | |
| 6 | CAGGTGACGTGTCTATAAAGAGTGTCAAATACCATTTAAT | linked marker |
| | ACTTTTAAACA | |
| 7 | AATGTTAGCTTATACAGATTCAAATTCAGGAGTTAGTCATT | linked marker |
| | TCCAAACAAG | |
| 8 | TTCACGTACTTAAAACCCAGATACGAGATATTTTACTAAG | linked marker |
| | CTATTGCTGTT | |
| 9 | TAATGAAATCTTGTAGTGCACAAATAGTCACACTTAACAT | linked marker |
| | CAAAGCAAAAT | |
| 10 | TTTTTAATAGCTCGTGGTATATCCATATCTTCTCTATACAA | linked marker |
| | TATACTTGAT | |
| 11 | TCAACAAGCGCATGAACCATAATCACAAGCATATACAAAC | linked marker |
| | CTAGTTATTTT | |
| 12 | TTACGCCCTTCAGCTTACTTGAGTTTGTTACAGGGTCGTT | linked marker |
| | CTTCTTTGAGG | |
| 13 | GCAACGTCCTCCCACTTTCTATTGATTTTACTGGTTAACC | linked marker |
| | AACTCAAGGTG | |
| 14 | CTCATTATTTTTATGGTTCTCTGTGTTTGCATCCACGTAGT | linked marker |
| | AGAAATAGAA | |
| 15 | GTGTTGAAGATATGACATCCTGGCGGAAACAACAGCTTC | linked marker |
| | AAACTGTTGCTA | |
| 16 | ATGCTATGGAGATGCAACTGGGTAAAGCAGAGGAGGAAA | linked marker |
| | CTCATCAACACA | |
| 17 | TCAGTGTCTTTTTATATGCTTGTTTATACTAGTTTTTAGCC | linked marker |
| | TTTGGAGTAG | |
| 18 | TTTGACTTCATGGTGATGCAACAAAGTTATTTTCTGCAGA | linked marker |
| | ATGTGAGAATT | |
| 19 | AACTCATTGACATACATTGAACATAAACAAACTTGCTTCT | linked marker |
| | GCACAAAAACG | |
| 20 | GAGTCATGCAGCGCAGAAATCTGTTAGTTTAACAGGTTC | linked marker |
| | GCAACCAGATGT | |
| 21 | CCTGATGCTTACTTGGAGTATTTTATATCAGTAATCGACG | linked marker |
| | CAGGCGGGATC | |
| 22 | GCTGTTACACTCAGGTGTATGCAAGCTCTTGTTCGTGTTC | linked marker |
| | AGTCTCGTGTA | |
| 23 | AGAAAGTCTCATTATACTTTGCTTCCCTTCCGGCGTTGGA | linked marker |
| | ATCACTGAAAT | |
| 24 | ATCGCCTTTATGTCATTCTTTCGCAGCTTAAAACATCACC | linked marker |
| | AAACGTTCTCA | |
| 25 | TCATGCATATGATCGGGTTCGAGACCCTGATTTCCAGTTT | linked marker |
| | CGGATTAGTTC | |
| 26 | GATATTTTTGCTTGGCTAACGTCTTAGTTTATATACTCGGT | linked marker |
| | GGTGGAGACG | |
| 27 | CTCAGTTGAGGCGTTAGTAATATTCGGCTGAGACAGCAT | linked marker |
| | GTTGGATTGAGT | |
| 28 | CGTGTCGTATTATAGAAGACGCAGCATTAGAGCAGCTCG | linked marker |
| | ATCCATTAGACT | |
| 29 | CTGGAGTAACATGAGCTGCTTTTTAGGATTCGACCCAATC | linked marker |
| | GATGGTACGTC | |

In parallel, F3 and BC1F1 seeds were sown for next round of infection and marker analysis. All of eight F3 families were resistant to Pb #0 confirming the resistance observed in F2. In addition, resistance was highly correlated with one (heterozygous) or both (homozygous) alleles from the BR-14 donor diagnosed by a KASP marker based on a marker as provided herein. The same observation was made with BC1F1 plants: only one of 23 plants had the susceptibility allele as detected by using a marker provided herein, but was resistant in the biotest. All other BC1F1 were heterozygous for a marker as provided herein corroborating the genetic stability of the resistance and its linkage to the QTL on chromosome A06. Resistant BC1F1 plants were selected to produce BC1F2 seeds, destined for further marker refinement in the BC1F2 generation.

In the present experiment, the markers according to SEQ ID Nos: 5, 6, 8, 13, 15, 19, 21, 22, 26 and 28 were monomorph in the F2 population.

Clubroot Resistance Intropressed from Br-14 Confers Broad Spectrum Resistance

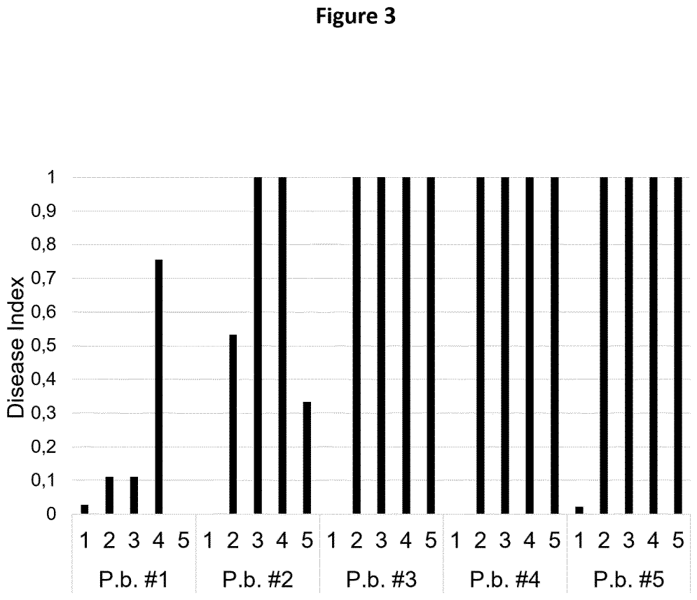

When testing the efficiency of the new resistance against Plasmodiophora brassicae isolates other than Pb #0, the inventors first infected plants of a previously resistant F3 family in separate bioassays with four additional isolates able to cause disease on MENDEL and extended (CRE1) resistance plus one isolate virulent on the susceptible control only. As a result, none of the tested Plasmodiophora brassicae isolates was able to infect a plant carrying the new CR allele (FIG. 3).

In a second experiment the inventors further extended the range of isolates by using different Plasmodiophora brassicae pathotypes identified in Canada (FIG. 4). Surprisingly, none of the tested isolates was able to cause clubroot disease on a line carrying the new resistance allele on chromosome A06. This was intriguing, since it indicated that the inventors gained with just a single locus a major part of the missing Br-14 resistance. With this embodiment the new locus on A06 confers a wide resistance against different isolates and pathotypes of Plasmodiophora brassicae.

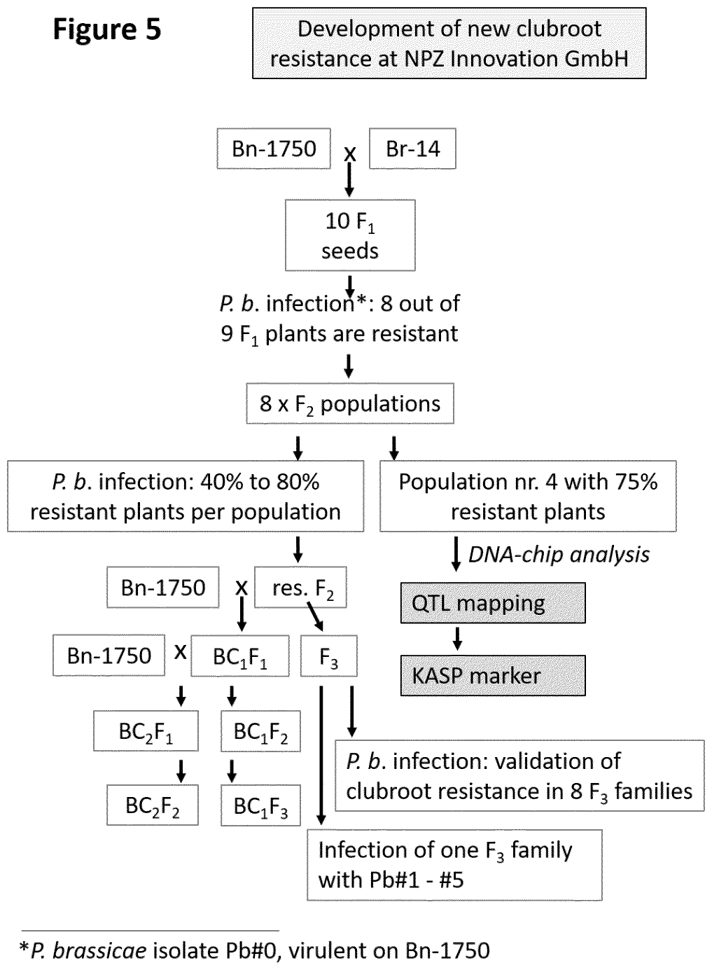

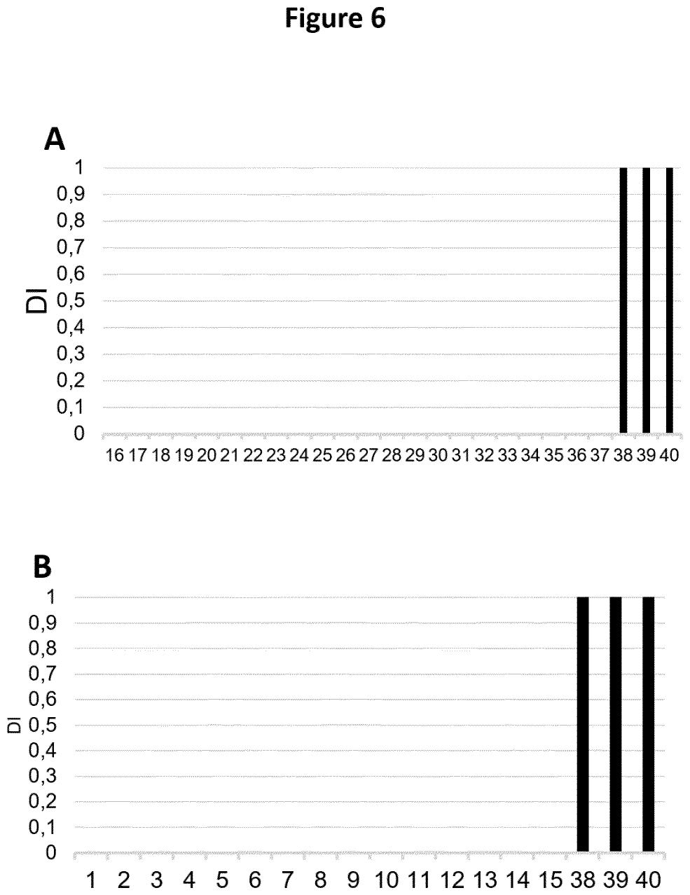

To further reduce unintended genomic background from Br-14 introgression resistant BC1F1 plants have been back-crossed to generate BC2F3. In parallel the said BC1F1 plants were selfed to set seed and subsequently generate BC1F3 plants. All progenies were analysed using a marker as provided herein. Surprisingly, the tested isolate Pb #0 was not able to cause clubroot disease on either BC2F3 lines or BC1F3 lines carrying the new resistance allele on chromosome A06 as analysed by a marker as provided herein (FIG. 5).

New CR is Linked to Molecular Marker in Advanced Backcross Progenies

An advanced backcross population BC2F2 (back cross 2 F2) produced independently of the mapping population, but from the same cross of Bn-1750 x Br-14 (Nr. 2 in Table 1) was used to confirm association of CR with one of the molecular markers identified for the QTL and biotests with Pb #0 and delivery, i.e. inheritance, to progenies. The molecular marker according to SEQ ID NO: 3 was exemplarily chosen for genotyping. Different F2 populations arising from the same cross can reflect independent Introgression events or Br-14 DNA into the genome of the F2 progeny. Of 187 BC2F2-plants tested, 138 individuals showed no disease symptoms and 49 plants were highly susceptible (Table 3). This follows a 3:1 segregation as was expected for a single dominant CR locus. Each plant was genotyped with the molecular marker according to SEQ ID NO: 3, and 137 resistant plants carried at least one resistance allele of Br-14. A disconnection between the prediction based on the molecular marker according to SEQ ID NO: 3 genotype and observed phenotype was observed in only three cases: Two susceptible plants were heterozygous for the marker according to SEQ ID NO: 3 and one resistant plant did not carry the corresponding allele of Br-14. We concluded that the markers according to SEQ ID Nos. 1-29 disclosed herein are tightly linked to new CR, exemplified by our proof-of-concept analysis with the marker according to SEQ ID NO: 3. Surprisingly, as shown using this second F2 population, the markers are linked to the new CR in plants derived from independent introgression of Br-14 DNA into B. napus genome.

TABLE 3
Segregation of new CR in a BC2F2 population with Bn-1750
as recurrent parent and independent from the mapping
population. Plants were infected with Pb#0 as described
above and genotyped with the marker according to SEQ
ID NO: 3 using KASP technology. Genotypes are given as
homozygous (hom.) or heterozygous (het.) states for a
SNP associated with resistance or susceptibility.

| | | Genotype SEQ ID NO. 3 | | |
|---|---|---|---|---|
| | | hom. res | het | hom. sus |
| Phenotype | susceptible | | 2 | 47 |
| | resistant | 49 | 88 | 1 |

Whole Genome Sequencing Reveals the Genomic Structure of CR

Characterization of the new CR on the genomic DNA level allows us to gain information on site and size of Br-14 introgression on the physical map of B. napus chromosome.

Subsequently, sequence information can be used for the identification of Br-14 specific sequence polymorphisms and the development of respective molecular markers that enable highly specific detection of the introgression in offspring genotypes from crosses with diverse genetic variability. Also, sequence in the QTL region can be explored for variants of defense-related genes that might be causative for new CR. To achieve sequence information on the QTL region we first compiled two bulks of plants of the BC2F2 population described in Table 3. Each bulk consisted of either 20 resistant or 20 susceptible plants. The plant samples in each bulk were pooled for DNA extraction using known methods. Prepared DNA was sent to a service provider for whole genome sequencing with Illumina HiSeq technology. To further characterize the CR, the CR donor Br-14 was individually sequenced as separate sample. In all cases, the amount of data retrieved from sequencing reflected about 40 times the size of B. napus genome (n=19) and the B. rapa genome (n=10) respectively per DNA sample. We applied a bioinformatics approach based on k-mer subtraction (as described by Prodhomme et al., 2019) to analyse sequence data from all resistant plants which could share a region comprising the new CR QTL and all susceptible plants lacking this region. Sequences of each pool were cut into 31 bp long k-mers and all k-mer sequences equally present in both pools were removed from the resistant plant pool. Left-over k-mers of the resistant pool were subsequently used to extract sequencing reads that match with the k-mer sequence from Br-14 and the resistant pool.

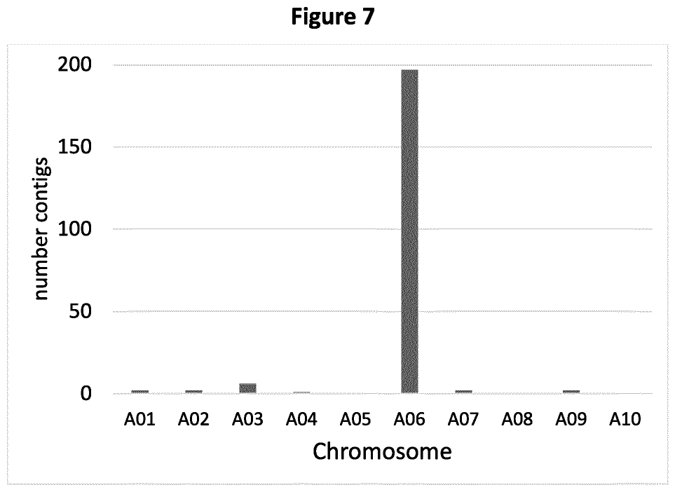

Localisation of these sequences in the B. napus genome were achieved through two strategies: 1. The extracted raw reads were mapped against reference genome Darmor-bzh (4.1) and 2. the extracted reads were de novo assembled to contigs (CLC Genomics Workbench, QIAGEN) and afterwards contigs aligned to the same reference genome by BLASTn algorithm (NCBI). The e-value of BLASTn was set to zero and only contigs were considered that gave a single hit in the genome. FIG. 7 shows how the contigs distribute among the ten A-genome chromosomes. By far the most contigs share homology with the sequence of chromosome A06.

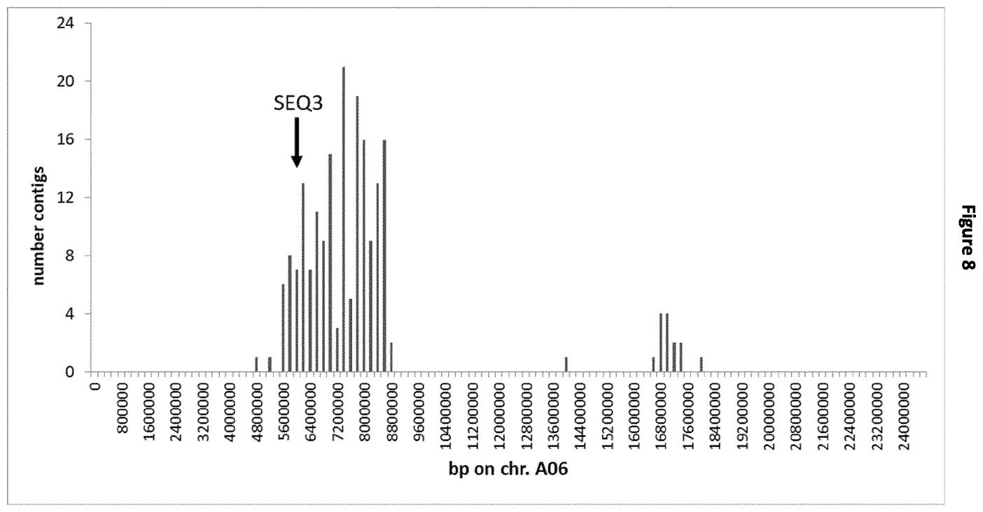

The A06 chromosome of Brassica napus has a length of about 24.4 Mbp and, as shown in FIG. 8, most of the contigs matching on chromosome A06 reside in a region between 4.6 Mbp and 8.6 Mbp. This confirms our initial results for the QTL of the F2 generation of population Nr. 4 which mapped to a region between 3.7 Mbp and 8.6 Mbp. We concluded that the new CR of BC2F2 population used in the sequencing approach originated from a Br-14 introgression between 4.6 Mbp and 8.6 Mbp of chromosome A06 into the B. napus genome and overlaps with the previously mapped QTL for the F2 population Nr. 4 above.

In order to retrieve sequence information from the original CR donor line Br-14 we performed a de novo assembly with all sequencing reads of Br-14 only and compared the resulting Br-14 contigs with reference genome B. napus var. Darmor-bzh version 4.1 using BLASTn algorithm (NCBI). According to QTL mapping and sequencing approaches, the physical position of new CR was set between 3.7 Mbp and 8.6 Mbp on chromosome A06. In this interval we identified 2757 Br-14 contigs matching with an e-value of 0.0 and distributed throughout the whole region. Among those Br-14 contigs we found sequences that are homologous to the sequences of the molecular markers identified herein, including SEQ ID NO. 1 to SEQ ID NO. 3.

Mapping of the CR specific sequencing reads derived from the k-mer selection revealed 1.218.276 sequencing reads that matched in the QTL region as well as in the introgression region on chromosome A06.

In the future, sequence information of the new CR QTL generated herein and possibly complemented with long-read sequence information will allow the identification of sequence variations between Br-14 and susceptible reference genomes like Brassica rapa v3.0. Subsequently, differential sequences can be explored for genes that have already been brought into context with plant defence or particularly plant defence against P. brassicae. These genes would represent candidate genes for new CR of Br-14 and to prove functionality, transfer of those genes into a susceptible Brassica plant individually could verify the link between candidate gene and new CR in a biotest.

### Embodiments

The invention is further described by the following embodiments:

1. A method for identifying or selecting a Brassica plant cell, Brassica plant or part thereof, or a Brassica seed comprising a QTL that confers resistance against clubroot disease, the method comprising
(i) detecting a QTL in the genome of the Brassica plant cell, Brassica plant or part thereof, or Brassica seed between a marker according to SEQ ID NO:1 and a marker according to SEQ ID NO:2; or
(ii) detecting a QTL in the genome of the Brassica plant cell, Brassica plant or part thereof, or Brassica seed linked to a marker according to any one of SEQ ID NO: 3 to SEQ ID NO: 29; or
(iii) detecting a QTL in the genome of the Brassica plant cell, Brassica plant or part thereof, or Brassica seed localizing within a genomic region, wherein the genomic region corresponds to position 3759027 to 8593901 of chromosome A06 of the Darmor reference genome.

2. The method according to embodiment 1, wherein the method involves the steps of
(a) obtaining a sample from the Brassica plant cell, the Brassica plant or part thereof, or the Brassica seed;
(b) isolating genomic DNA from the sample; and
(c) detecting the presence of the QTL in the genomic DNA.

3. The method of embodiment 1 or embodiment 2, wherein the detection of the QTL involves the detection of one or more markers linked to the QTL.

4. The method of embodiment 3, wherein the detection of the one or more markers occurs by PCR, hybridization, KASP assay, SNP analysis, genotyping, nucleic acid sequencing, next generation sequencing, nuclease based detection, by using an antibody, by using nucleic acid probes or by DNA chip technology.

5. The method according to embodiment 3 or embodiment 4, wherein the one or more markers linked to the QTL are within 50 cM of the QTL.

6. The method of any one of embodiments 1-5, wherein the resistance against clubroot disease is dominant.

7. The method of any one of embodiments 1-6, wherein the Brassica plant cell, Brassica plant or part thereof, or Brassica seed is Brassica napus.

8. The method of any one of embodiments 1-6, wherein the Brassica plant cell, Brassica plant or part thereof, or Brassica seed is Brassica rapa.

9. The method of any one of embodiments 1-6, wherein the Brassica plant cell, Brassica plant or part thereof, or Brassica seed is Brassica juncea.

10. The method of any one of embodiments 1-6, wherein the Brassica plant cell, Brassica plant or part thereof, or Brassica seed is Brassica carinata or Brassica oleracea.

11. The method of any one of embodiments 1-10, wherein the method is for determining the zygosity status of the Brassica plant cell, the Brassica plant or part thereof, or the Brassica seed with regard to the QTL.

12. A nucleic acid comprising a QTL that confers resistance against clubroot disease localizing within a genomic region of Brassica napus grown from seeds deposited under NCIMB 43700, wherein the genomic region corresponds to position 3759027 to 8593901 of chromosome A06 in the Darmor reference genome.

13. A nucleic acid comprising a QTL that confers resistance against clubroot disease, wherein the QTL has at least 70% sequence identity, at least 80% sequence identity, at least 90% sequence identity, at least 95% sequence identity or at least 99% sequence identity with a QTL localizing in Brassica napus grown from seeds deposited under NCIMB 43700 within a genomic region, wherein the genomic region corresponds to position 3759027 to 8593901 of chromosome A06 of the Darmor reference genome.

14. A nucleic acid comprising a QTL that confers resistance against clubroot disease localizing within a genomic region of the genome of a Brassica plant
(a) flanked by a marker according to SEQ ID NO:1 and a marker according to SEQ ID NO:2, or
(b) linked to a marker according to any one of SEQ ID NO: 3 to SEQ ID NO: 29.

15. A nucleic acid comprising a QTL that confers resistance against clubroot disease, wherein the QTL has at least 70% sequence identity, at least 80% sequence identity, at least 90% sequence identity, at least 95% sequence identity or at least 99% sequence identity to a QTL that confers resistance against clubroot disease localizing within a genomic region of the genome of a Brassica plant
(a) flanked by a marker according to SEQ ID NO:1 and a marker according to SEQ ID NO:2, or
(b) linked to a marker according to any one of SEQ ID NO: 3 to SEQ ID NO: 29.

16. The nucleic acid according to embodiment 14 or embodiment 15, wherein the Brassica plant is grown from seeds deposited under NCIMB 43700.

17. A nucleic acid comprising an open reading frame encoding a protein conferring resistance against clubroot disease, wherein the encoded protein sequence corresponds to an encoded protein sequence localizing within a genomic region of Brassica napus grown from seeds deposited under NCIMB 43700, wherein the genomic region corresponds to position 3759027 to 8593901 of chromosome A06 in the Darmor reference genome.

18. A protein encoded by the nucleic acid of any one of embodiments 12-17.

19. An antibody directed against the protein of embodiment 18.

20. A plant cell, plant or part thereof, or a seed identified or selected by the method of any one of embodiments 1-11.

21. The plant cell, plant or part thereof, or seed of embodiment 20, wherein the method involves the nucleic acid of any one of embodiments 12-17 or the antibody of embodiment 19.

22. A Brassica napus plant cell, a Brassica napus plant or part thereof, or a Brassica napus seed comprising a nucleic acid according to of any one of embodiments 12-17 or the protein of embodiment 18.

23. The Brassica napus plant cell, the Brassica napus plant or part thereof or the Brassica napus seed of embodiment 22, wherein the nucleic acid or the protein confers resistance against clubroot disease.

24. The Brassica napus plant cell, the Brassica napus plant or part thereof, or the Brassica napus seed of embodiment 22 or embodiment 23, wherein the Brassica napus plant cell, the Brassica napus plant or part thereof, or the Brassica napus seed is homozygous or heterozygous for the nucleic acid.

25. A Brassica napus plant cell, a Brassica napus plant or part thereof grown from seeds deposited under NCIMB 43700.

26. A descendant of the Brassica napus plant of embodiment 25.

27. A descendant of the Brassica napus plant of embodiment 26 obtained by crossing.

28. The descendant of embodiment 26 or embodiment 27, wherein the descendant comprises a nucleic acid according to of any one of embodiments 12-17.

29. The Brassica napus plant cell or the Brassica napus plant or part thereof of any one of embodiments 22-28, wherein the Brassica napus plant cell, the Brassica napus plant or part thereof is resistant against clubroot disease.

30. The Brassica napus seed of any one of embodiments 22-24, wherein a Brassica napus plant grown from the Brassica napus seed is resistant against clubroot disease.

31. Use of a plant according to any one of embodiments 20-29 or a plant grown from a seed according to embodiment 30 for breeding, where in the breeding is conventional breeding, pedigree breeding, crossing, self-pollination, doubling haploidy, single seed descent, backcrossing or breeding by genetic transformation or genome editing, targeted mutagenesis or non-targeted mutagenesis.

32. A harvested part of a Brassica napus plant according to any one of embodiments 20-29.

33. The harvested part of embodiment 32, wherein the harvested part is a feed product.

34. A method for increasing resistance against clubroot disease in a Brassica plant, wherein the method comprises a step of introducing the nucleic acid of any one of embodiments 12-17 into the Brassica plant.

35. Use of the nucleic acid of any one of embodiments 12-17 in a method for increasing the resistance against clubroot disease in a Brassica plant.

36. The method of embodiment 34 or the use of embodiment 35, wherein the method involves genome editing, genetic transformation, targeted mutagenesis or non-targeted mutagenesis.

37. The method of embodiment 34 or the use of embodiment 35, wherein the method involves breeding or crossing.

38. A method for identifying a marker linked to a QTL that confers resistance against clubroot disease, wherein the QTL is localized
(i) within a genomic region of a Brassica plant between a marker according to SEQ ID NO:1 and a marker according to SEQ ID NO:2; or
(ii) within a genomic region of a Brassica plant linked to a marker according to any one of SEQ ID NO: 3 to SEQ ID NO: 29, or
(iii) within a genomic region of a Brassica plant corresponding to position 3759027 to 8593901 of the Darmor reference genome.

39. The method of embodiment 38, wherein the marker is within 50 cM of the QTL.

40. A marker identified by the method of embodiment 38 or embodiment 39.

41. A method for transferring a resistance to clubroot disease to a descendant of a Brassica plant comprising the steps of:
(i) obtaining a first Brassica plant that is resistant to clubroot disease,
(ii) crossing the first Brassica plant of step (i) with a second Brassica plant,
(iii) selecting a descendant of the first and second Brassica plant that is resistant to clubroot disease according to the method of any one of embodiments 1-11,
wherein the first Brassica plant comprises a nucleic acid according to any one of embodiments 12-17.

42. A crushed Brassica seed comprising the nucleic acid according to of any one of embodiments 12-17.

43. A crushed Brassica seed, wherein a representative of said seed is deposited under NCIMB 43700.

44. A method for obtaining oil or meal products from a Brassica seed comprising the nucleic acid according to of any one of embodiments 12-17 or from a Brassica seed, wherein a representative of said seed is deposited under NCI MB 43700, wherein the method comprises the steps of
(i) crushing the Brassica seed, and
(ii) extracting the oil or meal products from the brassica seed.

Oil or meal products obtained by the method of embodiment 44.

### REFERENCES

Aigu Y., Cao T., Strelkov I. S. et al. Identification of winter and spring Brassica napus genotypes with partial resistance to Canadian isolates of Plasmodiophora brassicae. Canadian Journal of Plant Pathology (2020)
Broman K W, Wu H, Sen S, Churchill G A R/qtl: QTL mapping in experimental crosses. Bioinformatics 19:889-890 (2003)
Buczacki, S. T., H. Toxopeus, P. Mattusch, T. Johnston, G. Dixon, and L. Hobolth Study of physiologic specialization in Plasmodiophora brassicae: Proposals forattempted rationalization through an international approach. Trans. Br. Mycol. Soc. 65:295-303 (1975)
Chen J, Jing J, Zhan Z, Zhang T, Zhang C, et al. Identification of Novel QTLs for Isolate-Specific Partial Resistance to Plasmodiophora brassicae in Brassica rapa. PLoS ONE 8 (12) (2013)
Chu, M., Song, T., Falk, K. C. et al. Fine mapping of Rcr1 and analyses of its effect on transcriptome patterns during infection by Plasmodiophora brassicae. BMC Genomics 15, 1166 (2014)
Hatakeyama K, Suwabe K, Tomita R N, Kato T, Nunome T, et al. Identification and Characterization of Crr1a, a Gene for Resistance to Clubroot Disease (Plasmodiophora brassicae Woronin) in Brassica rapa L. PLoS ONE 8(1) (2013)
Hatakeyama, K., Niwa, T., Kato, T. et al. The tandem repeated organization of NB-LRR genes in the clubroot-resistant CRb locus in Brassica rapa L. Mol Genet Genomics 292, 397-405 (2017)
Kato T, Hatakeyama K, Fukino N, Matsumoto S. Fine mapping of the clubroot resistance gene CRb and development of a useful selectable marker in Brassica rapa. Breed Sci. 2013, 63(1):116-124.
Laila R, Park J I, Robin A H K, et al. Mapping of a novel clubroot resistance QTL using ddRAD-seq in Chinese cabbage (Brassica rapa L.). BMC Plant Biol. 2019; 19(1):13 (2019)
Manzanares-Dauleux, M., Delourme, R., Baron, F. et al. Mapping of one major gene and of QTLs involved in resistance to clubroot in Brassica napus. Theor Appl Genet 101, 885-891 (2000)
Pang W, Fu P, Li X, Zhan Z, Yu S and Piao Z Identification and Mapping of the Clubroot Resistance Gene CRd in Chinese Cabbage (Brassica rapa ssp. pekinensis). Front. Plant Sci. 9:653. (2018)
Piao, Z., Ramchiary, N. & Lim, Y. P. Genetics of Clubroot Resistance in Brassica Species. J Plant Growth Regul 28, 252-264 (2009)
Prodhomme, C., Esselink, D., Borm, T. et al. Comparative Subsequence Sets Analysis (CoSSA) is a robust approach to identify haplotype specific SNPs; mapping and pedigree analysis of a potato wart disease resistance gene Sen3. Plant Methods 15, 60 (2019)
Rahman H., Peng G., Yu F. et al. Genetics and breeding for clubroot resistance in Canadian spring canola (Brassica napus L.), Canadian Journal of Plant Pathology, 36:sup1, 122-134 (2014)
Rietz S, Strehlow B, Frauen M, Diederichsen E and Leckband G Extended clubroot resistance in rapeseed is conferred by the novel trait CRE1. Poste at 15th International Rapeseed Congress (2019)
Schwelm, A., Fogelqvist, J., Knaust, A. et al. The Plasmodiophora brassicae genome reveals insights in its life cycle and ancestry of chitin synthases. Sci Rep 5, 11153 (2015)
Tomita, H., Shimizu, M., Asad-ud Doullah, M. et al. Accumulation of quantitative trait loci conferring broad-spectrum clubroot resistance in Brassica oleracea. Mol Breeding 32, 889-900 (2013)
Ueno, H., Matsumoto, E., Aruga, D. et al. Molecular characterization of the CRa gene conferring clubroot resistance in Brassica rapa. Plant Mol Biol 80, 621-629 (2012)
Yu, F., Zhang, X., Peng, G. et al. Genotyping-by-sequencing reveals three QTL for clubroot resistance to six pathotypes of Plasmodiophora brassicae in Brassica rapa. Sci Rep 7, 4516 (2017)
Zhang, L., Cai, X., Wu, J. et al. Improved Brassica rapa reference genome by single-molecule sequencing and chromosome conformation capture technologies. Hortic Res 5, (2018). https://doi.org/10.1038/s41438-018-0071-9

1. A method for identifying or selecting a Brassica plant cell, Brassica plant or part thereof, or a Brassica seed comprising a QTL that confers resistance against clubroot disease, the method comprising
(i) detecting a QTL in the genome of the Brassica plant cell, Brassica plant or part thereof, or Brassica seed between a marker according to SEQ ID NO:1 and a marker according to SEQ ID NO:2; or
(ii) detecting a QTL in the genome of the Brassica plant cell, Brassica plant or part thereof, or Brassica seed linked to a marker according to any one of SEQ ID NO: 3 to SEQ ID NO: 29; or
(iii) detecting a QTL in the genome of the Brassica plant cell, Brassica plant or part thereof, or Brassica seed localizing within a genomic region, wherein the genomic region corresponds to position 3759027 to 8593901 of chromosome A06 of the Darmor reference genome.

2. The method according to claim 1, wherein the method involves the steps of
(a) obtaining a sample from the Brassica plant cell, the Brassica plant or part thereof, or the Brassica seed;
(b) isolating genomic DNA from the sample; and
(c) detecting the presence of the QTL in the genomic DNA.

3. The method of claim 1 or claim 2, wherein the detection of the QTL involves the detection of one or more markers linked to the QTL.

4. The method of claim 3, wherein the detection of the one or more markers occurs by PCR, hybridization, KASP assay, SNP analysis, genotyping, nucleic acid sequencing, next generation sequencing, nuclease based detection, by using an antibody, by using nucleic acid probes or by DNA chip technology.

5. The method according to claim 3 or claim 4, wherein the one or more markers linked to the QTL are within 50 cM of the QTL.

6. The method of any one of claims 1-5, wherein the Brassica plant cell, Brassica plant or part thereof, or Brassica seed is Brassica napus, Brassica rapa, Brassica juncea, Brassica carinata or Brassica oleracea.

7. The method of any one of claims 1-6, wherein the method is for determining the zygosity status of the Brassica plant cell, the Brassica plant or part thereof, or the Brassica seed with regard to the QTL.

8. A nucleic acid comprising a QTL that confers resistance against clubroot disease, wherein the QTL has at least 70% sequence identity, at least 80% sequence identity, at least 90% sequence identity, at least 95% sequence identity or at least 99% sequence identity to a QTL that confers resistance against clubroot disease localizing within a genomic region of the genome of a Brassica plant
(a) flanked by a marker according to SEQ ID NO:1 and a marker according to SEQ ID NO:2, or
(b) linked to a marker according to any one of SEQ ID NO: 3 to SEQ ID NO: 29,
optionally wherein the Brassica plant is grown from seeds deposited under NCIMB 43700.

9. A nucleic acid comprising an open reading frame encoding a protein conferring resistance against clubroot disease, wherein the encoded protein sequence corresponds to an encoded protein sequence localizing within a genomic region of Brassica napus grown from seeds deposited under NCIMB 43700, wherein the genomic region corresponds to position 3759027 to 8593901 of chromosome A06 in the Darmor reference genome.

10. A protein encoded by the nucleic acid of claim 8 or claim 9.

11. An antibody directed against the protein of claim 10.

12. Use of the nucleic acid of claim 8 or claim 9 in a method for increasing the resistance against clubroot disease in a Brassica plant

13. The use of claim 12, wherein the method involves genome editing, genetic transformation, targeted mutagenesis or non-targeted mutagenesis.

14. A method for identifying a marker linked to a QTL that confers resistance against clubroot disease, wherein the QTL is localized
(i) within a genomic region of a Brassica plant between a marker according to SEQ ID NO:1 and a marker according to SEQ ID NO:2; or
(ii) within a genomic region of a Brassica plant linked to a marker according to any one of SEQ ID NO: 3 to SEQ ID NO: 29, or
iii) within a genomic region of a Brassica plant corresponding to position 3759027 to 8593901 of the Darmor reference genome.

15. A marker identified by the method of claim 14.


SEQUENCE LISTING (29 sequences)

SEQ ID NO: 1 - DNA, 51 bp, Brassica napus
gttatctaggaatagagcttgaataaatgatagccatcttgtgatgaactt

SEQ ID NO: 2 - DNA, 51 bp, Brassica napus
ttctacgggtgtgataaatagatatctaaggtagagtccgtgagtttcatc

SEQ ID NO: 3 - DNA, 51 bp, Brassica napus
tgaaccctgattgttcttcacttcaaatatcacacagaagattgtatgcag

SEQ ID NO: 4 - DNA, 51 bp, Brassica napus
gtttgtgttttgtgaatcttaaatgcttaactaaggaaagttactaaaaag

SEQ ID NO: 5 - DNA, 51 bp, Brassica napus
cgcgtggcgaccatcttaattaaccggtttatcatccaatcgataagtttc

SEQ ID NO: 6 - DNA, 51 bp, Brassica napus
caggtgacgtgtctataaagagtgtcaaataccatttaatacttttaaaca

SEQ ID NO: 7 - DNA, 51 bp, Brassica napus
aatgttagcttatacagattcaaattcaggagttagtcatttccaaacaag

SEQ ID NO: 8 - DNA, 51 bp, Brassica napus
ttcacgtacttaaaacccagatacgagatattttactaagctattgctgtt

SEQ ID NO: 9 - DNA, 51 bp, Brassica napus
taatgaaatcttgtagtgcacaaatagtcacacttaacatcaaagcaaaat

SEQ ID NO: 10 - DNA, 51 bp, Brassica napus
tttttaatagctcgtggtatatccatatcttctctatacaatatacttgat

SEQ ID NO: 11 - DNA, 51 bp, Brassica napus
tcaacaagcgcatgaaccataatcacaagcatatacaaacctagttatttt

SEQ ID NO: 12 - DNA, 51 bp, Brassica napus
ttacgcccttcagcttacttgagtttgttacagggtcgttcttctttgagg

SEQ ID NO: 13 - DNA, 51 bp, Brassica napus
gcaacgtcctcccactttctattgattttactggttaaccaactcaaggtg

SEQ ID NO: 14 - DNA, 51 bp, Brassica napus
ctcattatttttatggttctctgtgtttgcatccacgtagtagaaatagaa

SEQ ID NO: 15 - DNA, 51 bp, Brassica napus
gtgttgaagatatgacatcctggcggaaacaacagcttcaaactgttgcta

SEQ ID NO: 16 - DNA, 51 bp, Brassica napus
atgctatggagatgcaactgggtaaagcagaggaggaaactcatcaacaca

SEQ ID NO: 17 - DNA, 51 bp, Brassica napus
tcagtgtctttttatatgcttgtttatactagtttttagcctttggagtag

SEQ ID NO: 18 - DNA, 51 bp, Brassica napus
tttgacttcatggtgatgcaacaaagttattttctgcagaatgtgagaatt

SEQ ID NO: 19 - DNA, 51 bp, Brassica napus
aactcattgacatacattgaacataaacaaacttgcttctgcacaaaaacg

SEQ ID NO: 20 - DNA, 51 bp, Brassica napus
gagtcatgcagcgcagaaatctgttagtttaacaggttcgcaaccagatgt

SEQ ID NO: 21 - DNA, 51 bp, Brassica napus
cctgatgcttacttggagtattttatatcagtaatcgacgcaggcgggatc

SEQ ID NO: 22 - DNA, 51 bp, Brassica napus
gctgttacactcaggtgtatgcaagctcttgttcgtgttcagtctcgtgta

SEQ ID NO: 23 - DNA, 51 bp, Brassica napus
agaaagtctcattatactttgcttcccttccggcgttggaatcactgaaat

SEQ ID NO: 24 - DNA, 51 bp, Brassica napus
atcgcctttatgtcattctttcgcagcttaaaacatcaccaaacgttctca

SEQ ID NO: 25 - DNA, 51 bp, Brassica napus
tcatgcatatgatcgggttcgagaccctgatttccagtttcggattagttc

SEQ ID NO: 26 - DNA, 51 bp, Brassica napus
gatatttttgcttggctaacgtcttagtttatatactcggtggtggagacg

SEQ ID NO: 27 - DNA, 51 bp, Brassica napus
ctcagttgaggcgttagtaatattcggctgagacagcatgttggattgagt

SEQ ID NO: 28 - DNA, 51 bp, Brassica napus
cgtgtcgtattatagaagacgcagcattagagcagctcgatccattagact

SEQ ID NO: 29 - DNA, 51 bp, Brassica napus
ctggagtaacatgagctgctttttaggattcgacccaatcgatggtacgtc